SEQUENCE LISTING (13 sequences)

SEQ ID NO: 1 - protein, 1174 aa, Mycobacterium marinum
MSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 2 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 3 - protein, 1148 aa, Segniliparus rugosus
MGDGEERAKRFFQRIGELSATDPQFAAAAPDPAVVEAVSDPSLSFTRYLDTLMRGYAERPALAHRVGAGYETISYGELWARVGAIAAAWQADGLAPGDFVATVGFTSPDYVAVDLAAARSGLVSVPLQAGASLAQLVGILEETEPKVLAASASSLEGAVACALAAPSVQRLVVFDLRGPDASESAADERRGALADAEEQLARAGRAVVVETLADLAARGEALPEAPLFEPAEGEDPLALLIYTSGSTGAPKGAMYSQRLVSQLWGRTPVVPGMPNISLHYMPLSHSYGRAVLAGALSAGGTAHFTANSDLSTLFEDIALARPTFLALVPRVCEMLFQESQRGQDVAELRERVLGGRLLVAVCGSAPLSPEMRAFMEEVLGFPLLDGYGSTEALGVMRNGIIQRPPVIDYKLVDVPELGYRTTDKPYPRGELCIRSTSLISGYYKRPEITAEVFDAQGYYKTGDVMAEIAPDHLVYVDRSKNVLKLSQGEFVAVAKLEAAYGTSPYVKQIFVYGNSERSFLLAVVVPNAEVLGARDQEEAKPLIAASLQKIAKEAGLQSYEVPRDFLIETEPFTTQNGLLSEVGKLLRPKLKARYGEALEARYDEIAHGQADELRALRDGAGQRPVVETVVRAAVAISGSEGAEVGPEANFADLGGDSLSALSLANLLHDVFEVEVPVRIIIGPTASLAGIAKHIEAERAGASAPTAASVHGAGATRIRASELTLEKFLPEDLLAAAKGLPAADQVRTVLLTGANGWLGRFLALEQLERLARSGQDGGKLICLVRGKDAAAARRRIEETLGTDPALAARFAELAEGRLEVVPGDVGEPKFGLDDAAWDRLAEEVDVIVHPAALVNHVLPYHQLFGPNVVGTAEIIRLAITAKRKPVTYLSTVAVAAGVEPSSFEEDGDIRAVVPERPLGDGYANGYGNSKWAGEVLLREAHELVGLPVAVFRSDMILAHTRYTGQLNVPDQFTRLVLSLLATGIAPKSFYQQGAAGERQRAHYDGIPVDFTAEAITTLGAEPSWFDGGAGFRSFDVFNPHHDGVGLDEFVDWLIEAGHPISRIDDHKEWFARFETAVRGLPEAQRQHSLLPLLRAYSFPHPPVDGSVYPTGKFQGAVKAAQVGSDHDVPHLGKALIVKYADDLKALGLL

SEQ ID NO: 4 - protein, 222 aa, Nocardia sp. NRRL 5646
MIETILPAGVESAELLEYPEDLKAHPAEEHLIAKSVEKRRRDFIGARHCARLALAELGEPPVAIGKGERGAPIWPRGVVGSLTHCDGYRAAAVAHKMRFRSIGIDAEPHATLPEGVLDSVSLPPEREWLKTTDSALHLDRLLFCAKEATYKAWWPLTARWLGFEEAHITFEIEDGSADSGNGTFHSELLVPGQTNDGGTPLLSFDGRWLIADGFILTAIAYA

SEQ ID NO: 5 - protein, 1185 aa, Mycobacterium massiliense
MTNETNPQQEQLSRRIESLRESDPQFRAAQPDPAVAEQVLRPGLHLSEAIAALMTGYAERPALGERARELVIDQDGRTTLRLLPRFDTTTYGELWSRTTSVAAAWHHDATHPVKAGDLVATLGFTSIDYTVLDLAIMILGGVAVPLQTSAPASQWTTILAEAEPNTLAVSIELIGAAMESVRATPSIKQVVVFDYTPEVDDQREAFEAASTQLAGTGIALETLDAVIARGAALPAAPLYAPSAGDDPLALLIYTSGSTGAPKGAMHSENIVRRWWIREDVMAGTENLPMIGLNFMPMSHIMGRGTLTSTLSTGGTGYFAASSDMSTLFEDMELIRPTALALVPRVCDMVFQRFQTEVDRRLASGDTASAEAVAAEVKADIRDNLFGGRVSAVMVGSAPLSEELGEFIESCFELNLTDGYGSTEAGMVFRDGIVQRPPVIDYKLVDVPELGYFSTDKPHPRGELLLKTDGMFLGYYKRPEVTASVFDADGFYMTGDIVAELAHDNIEIIDRRNNVLKLSQGEFVAVATLEAEYANSPVVHQIYVYGSSERSYLLAVVVPTPEAVAAAKGDAAALKTTIADSLQDIAKEIQLQSYEVPRDFIIEPQPFTQGNGLLTGIAKLARPNLKAHYGPRLEQMYAEIAEQQAAELRALHGVDPDKPALETVLKAAQALLGVSSAELAADAHFTDLGGDSLSALSFSDLLRDIFAVEVPVGVIVSAANDLGGVAKFVDEQRHSGGTRPTAETVHGAGHTEIRAADLTLDKFIDEATLHAAPSLPKAAGIPHTVLLTGSNGYLGHYLALEWLERLDKTDGKLIVIVRGKNAEAAYGRLEEAFDTGDTELLAHFRSLADKHLEVLAGDIGDPNLGLDADTWQRLADTVDVIVHPAALVNHVLPYNQLFGPNVVGTAEIIKLAITTKIKPVTYLSTVAVAAYVDPTTFDEESDIRLISAVRPIDDGYANGYGNAKWAGEVLLREAHDLCGLPVAVFRSDMILAHSRYTGQLNVPDQFTRLILSLIATGIAPGSFYQAQTTGERPLAHYDGLPGDFTAEAITTLGTQVPEGSEGFVTYDCVNPHADGISLDNFVDWLIEAGYPIARIDNYTEWFTRFDTAIRGLSEKQKQHSLLPLLHAFEQPSAAENHGVVPAKRFQHAVQAAGIGPVGQDGTTDIPHLSRRLIVKYAKDLEQLGLL

SEQ ID NO: 6 - protein, 1186 aa, Segniliparus rotundus
MTQSHTQGPQASAAHSRLARRAAELLATDPQAAATLPDPEVVRQATRPGLRLAERVDAILSGYADRPALGQRSFQTVKDPITGRSSVELLPTFDTITYRELRERATAIASDLAHHPQAPAKPGDFLASIGFISVDYVAIDIAGVFAGLTAVPLQTGATLATLTAITAETAPTLFAASIEHLPTAVDAVLATPSVRRLLVFDYRAGSDEDREAVEAAKRKIADAGSSVLVDVLDEVIARGKSAPKAPLPPATDAGDDSLSLLIYTSGSTGTPKGAMYPERNVAHFWGGVWAAAFDEDAAPPVPAINITFLPLSHVASRLSLMPTLARGGLMHFVAKSDLSTLFEDLKLARPTNLFLVPRVVEMLYQHYQSELDRRGVQDGTREAEAVKDDLRTGLLGGRILTAGFGSAPLSAELAGFIESLLQIHLVDGYGSTEAGPVWRDGYLVKPPVTDYKLIDVPELGYFSTDSPHPRGELAIKTQTILPGYYKRPETTAEVFDEDGFYLTGDVVAQIGPEQFAYVDRRKNVLKLSQGEFVTLAKLEAAYSSSPLVRQLFVYGSSERSYLLAVIVPTPDALKKFGVGEAAKAALGESLQKIARDEGLQSYEVPRDFIIETDPFTVENGLLSDARKSLRPKLKEHYGERLEAMYKELADGQANELRDIRRGVQQRPTLETVRRAAAAMLGASAAEIKPDAHFTDLGGDSLSALTFSNFLHDLFEVDVPVGVIVSAANTLGSVAEHIDAQLAGGRARPTFATVHGKGSTTIKASDLTLDKFIDEQTLEAAKHLPKPADPPRTVLLTGANGWLGRFLALEWLERLAPAGGKLITIVRGKDAAQAKARLDAAYESGDPKLAGHYQDLAATTLEVLAGDFSEPRLGLDEATWNRLADEVDFISHPGALVNHVLPYNQLFGPNVAGVAEIIKLAITTRIKPVTYLSTVAVAAGVEPSALDEDGDIRTVSAERSVDEGYANGYGNSKWGGEVLLREAHDRTGLPVRVFRSDMILAHQKYTGQVNATDQFTRLVQSLLATGLAPKSFYELDAQGNRQRAHYDGIPVDFTAESITTLGGDGLEGYRSYNVFNPHRDGVGLDEFVDWLIEAGHPITRIDDYDQWLSRFETSLRGLPESKRQASVLPLLHAFARPGPAVDGSPFRNTVFRTDVQKAKIGAEHDIPHLGKALVLKYADDIKQLGLL

SEQ ID NO: 7 - protein, 459 aa, Chromobacterium violaceum
MQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 8 - protein, 468 aa, Pseudomonas aeruginosa
MNARLHATSPLGDADLVRADQAHYMHGYHVFDDHRVNGSLNIAAGDGAYIYDTAGNRYLDAVGGMWCTNIGLGREEMARTVAEQTRLLAYSNPFCDMANPRAIELCRKLAELAPGDLDHVFLTTGGSTAVDTAIRLMHYYQNCRGKRAKKHVITRINAYHGSTFLGMSLGGKSADRPAEFDFLDERIHHLACPYYYRAPEGLGEAEFLDGLVDEFERKILELGADRVGAFISEPVFGSGGVIVPPAGYHRRMWELCQRYDVLYISDEVVTSFGRLGHFFASQAVFGVQPDIILTAKGLTSGYQPLGACIFSRRIWEVIAEPDKGRCFSHGFTYSGHPVACAAALKNIEIIEREGLLAHADEVGRYFEERLQSLRDLPIVGDVRGMRFMACVEFVADKASKALFPESLNIGEWVHLRAQKRGLLVRPIVHLNVMSPPLILTREQVDTVVRVLRESIEETVEDLVRAGHR

SEQ ID NO: 9 - protein, 454 aa, Pseudomonas syringae
MSANNPQTLEWQALSSEHHLAPFSDYKQLKEKGPRIITRAEGVYLWDSEGNKILDGMSGLWCVAIGYGREELADAASKQMRELPYYNLFFQTAHPPVLELAKAISDIAPEGMNHVFFTGSGSEGNDTMLRMVRHYWALKGQPNKKTIISRVNGYHGSTVAGASLGGMTYMHEQGDLPIPGVVHIPQPYWFGEGGDMTPDEFGIWAAEQLEKKILELGVENVGAFIAEPIQGAGGVIVPPDSYWPKIKEILSRYDILFAADEVICGFGRTSEWFGSDFYGLRPDMMTIAKGLTSGYVPMGGLIVRDEIVAVLNEGGDFNHGFTYSGHPVAAAVALENIRILREEKIVERVRSETAPYLQKRLRELSDHPLVGEVRGVGLLGAIELVKDKTTRERYTDKGAGMICRTFCFDNGLIMRAVGDTMIIAPPLVISFAQIDELVEKARTCLDLTLAVLQG

SEQ ID NO: 10 - protein, 467 aa, Rhodobacter sphaeroides
MTRNDATNAAGAVGAAMRDHILLPAQEMAKLGKSAQPVLTHAEGIYVHTEDGRRLIDGPAGMWCAQVGYGRREIVDAMAHQAMVLPYASPWYMATSPAARLAEKIATLTPGDLNRIFFTTGGSTAVDSALRFSEFYNNVLGRPQKKRIIVRYDGYHGSTALTAACTGRTGNWPNFDIAQDRISFLSSPNPRHAGNRSQEAFLDDLVQEFEDRIESLGPDTIAAFLAEPILASGGVIIPPAGYHARFKAICEKHDILYISDEVVTGFGRCGEWFASEKVFGVVPDIITFAKGVTSGYVPLGGLAISEAVLARISGENAKGSWFTNGYTYSNQPVACAAALANIELMEREGIVDQAREMADYFAAALASLRDLPGVAETRSVGLVGCVQCLLDPTRADGTAEDKAFTLKIDERCFELGLIVRPLGDLCVISPPLIISRAQIDEMVAIMRQAITEVSAAHGLTAKEPAAV

SEQ ID NO: 11 - protein, 459 aa, Escherichia coli
MNRLPSSASALACSAHALNLIEKRTLDHEEMKALNREVIEYFKEHVNPGFLEYRKSVTAGGDYGAVEWQAGSLNTLVDTQGQEFIDCLGGFGIFNVGHRNPVVVSAVQNQLAKQPLHSQELLDPLRAMLAKTLAALTPGKLKYSFFCNSGTESVEAALKLAKAYQSPRGKFTFIATSGAFHGKSLGALSATAKSTFRKPFMPLLPGFRHVPFGNIEAMRTALNECKKTGDDVAAVILEPIQGEGGVILPPPGYLTAVRKLCDEFGALMILDEVQTGMGRTGKMFACEHENVQPDILCLAKALGGGVMPIGATIATEEVFSVLFDNPFLHTTTFGGNPLACAAALATINVLLEQNLPAQAEQKGDMLLDGFRQLAREYPDLVQEARGKGMLMAIEFVDNEIGYNFASEMFRQRVLVAGTLNNAKTIRIEPPLTLTIEQCELVIKAARKALAAMRVSVEEA

SEQ ID NO: 12 - protein, 453 aa, Vibrio Fluvialis
MNKPQSWEARAETYSLYGFTDMPSLHQRGTVVVTHGEGPYIVDVNGRRYLDANSGLWNMVAGFDHKGLIDAAKAQYERFPGYHAFFGRMSDQTVMLSEKLVEVSPFDSGRVFYTNSGSEANDTMVKMLWFLHAAEGKPQKRKILTRWNAYHGVTAVSASMTGKPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPMGAVILGPELSKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICRPLGQSVVLCPPFILTEAQMDEMFDKLEKALDKVFAEVA

SEQ ID NO: 13 - protein, 224 aa, Bacillus subtilis
MKIYGIYMDRPLSQEENERFMSFISPEKREKCRRFYHKEDAHRTLLGDVLVRSVISRQYQLDKSDIRFSTQEYGKPCIPDLPDAHFNISHSGRWVICAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

(19) United States
(12) Patent Application Publication
(10) Pub. No.: US 2015/0111262 A1
(43) Pub. Date: Apr. 23, 2015

(54) METHODS AND MATERIALS FOR PRODUCING FIVE CARBON BUILDING BLOCKS FROM PROLINE

(71) Applicant: INVISTA North America S.á r.l., Wilmington, DE (US)

(72) Inventors: Adriana Leonora Botes, Rosedale East (GB); Alex Van Eck Conradie, Eaglescliffe (GB)

(21) Appl. No.: 14/470,698

(22) Filed: Aug. 27, 2014

Related U.S. Application Data
(60) Provisional application No. 61/870,438, filed on Aug. 27, 2013.
(60) Provisional application No. 62/012,608, filed on Jun. 16, 2014.

(51) Int. Cl. C12P 13/00; C12P 7/42; C12P 7/18; C12P 7/46

(52) U.S. Cl. CPC C12P 13/001; C12P 7/46; C12P 7/42; C12P 7/18
USPC 435/128; 435/145; 435/146; 435/158; 435/252.3; 435/252.33; 435/252.32; 435/252.34; 435/252.31; 435/254.11; 435/254.2; 435/254.3; 435/254.21; 435/254.23

(57) ABSTRACT

This document describes biochemical pathways for producing glutaric acid, 5-aminopentanoic acid, 5-hydroxypentanoic acid, cadaverine or 1,5-pentanediol by forming one or two terminal functional groups, comprised of carboxyl, amine or hydroxyl group, in a C5 backbone substrate such as D-proline.

### CROSS-REFERENCE TO RELATED APPLICATIONS

This application claims priority to U.S. application Ser. No. 61/870,438, filed Aug. 27, 2013, and to U.S. application Ser. No. 62/012,608, filed on Jun. 16, 2014. The disclosures of the applications are incorporated by reference in their entirety.

### TECHNICAL FIELD

This invention relates to methods for biosynthesizing one or more C5 building blocks selected from the group glutaric acid, 5-aminopentanoic acid, cadaverine, 5-hydroxypentanoic acid, and 1,5-pentanediol using one or more isolated enzymes such as reductases, racemases, kinases, dehydrogenases, or ω-transaminases, and recombinant hosts that produce such C5 building blocks.

### BACKGROUND

Cadaverine (1,5-pentanediamine) is a C5 diamine that can be used as a diamine monomer together with a diacid for polyamide synthesis such as nylon 5,10 or nylon 5,6. Cadaverine typically is produced by decarboxylation of lysine to cadaverine. See, for example, Qian et al., Biotechnol Bioeng. 108(1):93-103 (2011). Decarboxylation of lysine is not an efficient process, however, as two carbons are lost as CO₂ in this pathway (diamonopimelate decarboxylase (C7)→lysine decarboxylase (C6)→cadaverine). Accordingly, it is clear that there is a need for sustainable and efficient methods for producing cadaverine and other C5 monomers that can be used for producing polymers.

### SUMMARY

This document is based at least in part on the discovery that it is possible to construct biochemical pathways for producing a five carbon chain backbone precursor such as D-proline, in which one or two functional groups, i.e., carboxyl, amine or hydroxyl, can be formed, leading to the synthesis of one or more of glutarate, 5-hydroxypentanoate, 5-aminopentanoate (also known as 5-aminovalerate), cadaverine (also known as 1,5 pentanediamine), and 1,5-pentanediol (hereafter collectively referred to as “C5 building blocks” and each of the compounds being a “C5 building block”). Glutarate semialdehyde (also known as 5-oxopentanoic acid) can be produced as an intermediate to other products. Glutaric acid and glutarate, 5-hydroxypentanoic acid and 5-hydroxypentanoate, 5-oxopentanoic acid and 5-oxopentanoate, and 5-aminopentanoic and 5-aminopentanoate are used interchangeably herein to refer to the compound in any of its neutral or ionized forms, including any salt forms thereof. It is understood by those skilled in the art that the specific form will depend on pH.

In one aspect, this document features a method of producing a C5 building block selected from the group consisting of glutarate, 5-aminopentanoate, cadaverine, 5-hydroxpentanoate, and 1,5-pentanediol. The method includes enzymatically synthesizing D-proline and enzymatically converting D-proline to the C5 building block in one or more enzymatic steps. D-proline can be enzymatically synthesized from L-glutamic acid (e.g., using one or more such as one, two, three, or four of the following exogenous enzymes: a glutamate 5-kinase, a glutamate semialdehyde dehydrogenase, a pyrroline 5-carboxylate reductase, and a proline racemase).

D-proline can be enzymatically converted to 5-aminopentanoate using, for example, a D-proline reductase.

D-proline can be enzymatically converted to glutarate using, for example, a (i) D-proline reductase; (ii) a 5-aminovalerate transaminase; and (iii) a dehydrogenase selected from the group consisting of a glutarate semialdehyde dehydrogenase, a succinate-semialdehyde dehydrogenase, an aldehyde dehydrogenase, a 5-oxovalerate dehydrogenase, a 6-oxohexanoate dehydrogenase, and a 7-oxoheptanoate dehydrogenase. The 5-aminovalerate transaminase can have at least 70% sequence identity to the amino acid sequence set forth in SEQ ID NO:7 or SEQ ID NO:9.

D-proline can be enzymatically converted to 5-hydroxypentanoate using, for example, (i) a D-proline reductase; (ii) a 5-aminovalerate transaminase; and (iii) a dehydrogenase selected from the group consisting of a 6-hydroxyhexanoate dehydrogenase, a 5-hydroxypentanoate dehydrogenase, and a 4-hydroxybutyrate dehydrogenase. The 5-aminovalerate transaminase can have at least 70% sequence identity to the amino acid sequence set forth in SEQ ID NO:7 or SEQ ID NO:9. 5-hydroxypentanoate can be converted to cadaverine using a carboxylate reductase, an alcohol dehydrogenase, and at least one ω-transaminase (e.g., one ω-transaminase or two different ω-transaminases). The at least one ω-transaminase can be classified under EC 2.6.1.18, EC 2.6.1.19, EC 2.6.1.29, 2.6.1.48, or EC 2.6.1.82. The at least one ω-transaminase can have 70% or more sequence identity to an amino acid sequence set forth in SEQ ID NO: 7, SEQ ID NO: 8, SEQ ID NO: 9, SEQ ID NO: 10, SEQ ID NO: 11, or SEQ ID NO: 12. The carboxylate reductase can have at least 70% sequence identity to the amino acid sequence set forth in SEQ ID NO:1, SEQ ID NO:2, SEQ ID NO:3, SEQ ID NO:5, or SEQ ID NO:6.

5-hydroxypentanoate can be converted to 1,5-pentanediol using, for example, a carboxylate reductase and an alcohol dehydrogenase. The carboxylate reductase can have at least 70% sequence identity to the amino acid sequence set forth in SEQ ID NO:1, SEQ ID NO:2, SEQ ID NO:3, SEQ ID NO:5, or SEQ ID NO:6.

1,5-pentanediol can be enzymatically converted to cadaverine using an alcohol dehydrogenase and at least one ω-transaminase (e.g., one ω-transaminase or two different ω-transaminases). The at least one ω-transaminase can have at least 70% sequence identity to an amino acid sequence set forth in SEQ ID NO: 7, SEQ ID NO: 8, SEQ ID NO: 9, SEQ ID NO: 10, SEQ ID NO: 11, or SEQ ID NO: 12.

5-aminopentanoate can be enzymatically converted to cadaverine using, for example, a carboxylate reductase and a ω-transaminase. The ω-transaminase can have at least 70% sequence identity to an amino acid sequence set forth in SEQ ID NO: 7, SEQ ID NO: 8, SEQ ID NO: 9, or SEQ ID NO: 10.

D-proline can be enzymatically converted to cadaverine using a D-proline reductase, a 5-aminovalerate transaminase, an alcohol dehydrogenase, a carboxylate reductase, and at least one ω-transaminase. The 5-aminovalerate transaminase can have at least 70% sequence identity to the amino acid sequence set forth in SEQ ID NO:7 or SEQ ID NO:9. The carboxylate reductase can have at least 70% sequence identity to the amino acid sequence set forth in SEQ ID NO:6.

In any of the methods, all or part of the method can be performed in a recombinant host by fermentation. The host can be subjected to a cultivation strategy under aerobic, anaerobic or, micro-aerobic cultivation conditions. The host can be cultured under conditions of nutrient limitation. The host can be retained using a ceramic hollow fiber membrane to maintain a high cell density during fermentation. The principal carbon source fed to the fermentation can derive from a biological feedstock. For example, the biological feedstock can be, or can derive from, monosaccharides, disaccharides, lignocellulose, hemicellulose, cellulose, lignin, levulinic acid, formic acid, triglycerides, glycerol, fatty acids, agricultural waste, condensed distillers' solubles, or municipal waste. The principal carbon source fed to the fermentation can derive from a non-biological feedstock. For example, the non-biological feedstock can be, or can derive from, natural gas, syngas, CO₂/H₂, methanol, ethanol, benzoate, non-volatile residue (NVR) caustic wash waste stream from cyclohexane oxidation processes, or terephthalic acid/isophthalic acid mixture waste streams.

This document also features a recombinant host that includes at least one exogenous nucleic acid encoding (i) a D-proline reductase, (ii) a 5-aminovalerate transaminase, and (iii) a dehydrogenase, the host producing glutarate or 5-hydroxypentanoate. For example, the host can produce glutarate and the dehydrogenase can be selected from the group consisting of glutarate semialdehyde dehydrogenase, a succinate-semialdehyde dehydrogenase, an aldehyde dehydrogenase, a 5-oxovalerate dehydrogenase, a 6-oxohexanoate dehydrogenase, and a 7-oxoheptanoate dehydrogenase. For example, the host can produce 5-hydroxypentanoate and the dehydrogenase can be selected from the group consisting of a 6-hydroxyhexanoate dehydrogenase, a 5-hydroxypentanoate dehydrogenase, and a 4-hydroxybutyrate dehydrogenase. A host producing 5-hydroxypentanoate further can include an exogenous carboxylate reductase and an exogenous alcohol dehydrogenase, and further produce 1,5-pentanediol. A host producing 1,5-pentanediol further can include at least one exogenous ω-transaminase and an optional second and/or third exogenous alcohol dehydrogenase, and further produce cadaverine. A host producing 5-hydroxypentanoate further can include an exogenous carboxylate reductase, an exogenous alcohol dehydrogenase, and at least one exogenous ω-transaminase, and further produce cadaverine. The at least one exogenous ω-transaminase in any of the hosts can have at least 70% sequence identity to an amino acid sequence set forth in SEQ ID NO: 7, SEQ ID NO: 8, SEQ ID NO: 9, SEQ ID NO: 10, SEQ ID NO:11, or SEQ ID NO:12. The at least one exogenous ω-transaminase can be two different exogenous ω-transaminases. The 5-aminovalerate transaminase in any of the hosts can have at least 70% sequence identity to the amino acid sequence set forth in SEQ ID NO:7 or SEQ ID NO:9.

This document also features a recombinant host that includes at least one exogenous nucleic acid encoding (i) a D-proline reductase, (ii) a carboxylate reductase, and (iii) a ω-transaminase, the host producing cadaverine. The ω-transaminase can have at least 70% sequence identity to an amino acid sequence set forth in SEQ ID NO: 7, SEQ ID NO: 8, SEQ ID NO: 9, or SEQ ID NO: 10. The host further can include an exogenous 5-aminovalerate transaminase. The 5-aminovalerate transaminase can have at least 70% sequence identity to the amino acid sequence set forth in SEQ ID NO:7 or SEQ ID NO:9. The carboxylate reductase can have at least 70% sequence identity to the amino acid sequence set forth in SEQ ID NO:6.

This document also features a recombinant host that includes at least one exogenous nucleic acid encoding (i) a 5-aminovalerate transaminase and (ii) a dehydrogenase, the host producing glutarate or 5-hydroxypentanoate. The host further can include an exogenous carboxylate reductase and an exogenous alcohol dehydrogenase and further produce 1,5-pentanediol. The host producing 1,5-pentanediol further can include at least one exogenous ω-transaminase and an optional second or third alcohol dehydrogenase, and further produce cadaverine.

This document also features a recombinant host that includes at least one exogenous nucleic acid encoding (i) a carboxylate reductase and (ii) a ω-transaminase, the host producing cadaverine.

This document also features a recombinant host that includes at least one exogenous nucleic acid encoding (i) a carboxylate reductase, (ii) at least one ω-transaminase, and (iii) an alcohol dehydrogenase, the host producing cadaverine.

In any of the recombinant hosts, the carboxylate reductase can have at least 70% sequence identity to the amino acid sequence set forth in SEQ ID NO:1, SEQ ID NO:2, SEQ ID NO:3, SEQ ID NO:5, or SEQ ID NO:6.

Any of the recombinant hosts or any of the recombinant hosts used in any of the methods can be a prokaryote. The prokaryote can be from the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia acidovorans, from the genus Bacillus such as Bacillus subtillis; from the genes Lactobacillus such as Lactobacillus delbrueckii; from the genus Lactococcus such as Lactococcus lactis or from the genus Rhodococcus such as Rhodococcus equi.

Any of the recombinant hosts or any of the recombinant hosts used in any of the methods can be a eukaryote. The eukaryote can be from the genus Aspergillus such as Aspergillus niger; from the genus Saccharomyces such as Saccharomyces cerevisiae; from the genus Pichia such as Pichia pastoris; from the genus Yarrowia such as Yarrowia lipolytica, from the genus Issatchenkia such as Issathenkia orientalis, from the genus Debaryomyces such as Debaryomyces hansenii, from the genus Arxula such as Arxula adenoinivorans, or from the genus Kluyveromyces such as Kluyveromyces lactis.

The recombinant host or recombinant host used in any of the methods can include one or more of the following attenuated enzymes: a polyhydroxyalkanoate synthase; a triose phosphate isomerase; a glucose-6-phosphate isomerase; a transhydrogenase; an NADH-specific glutamate dehydrogenase; a NADH/NADPH-utilizing glutamate dehydrogenase; a glutaryl-CoA dehydrogenase; or a glutaryl-CoA synthetase.

Any of the recombinant hosts or any of the recombinant hosts used in any of the methods can overexpress one or more genes encoding: a phosphoenolpyruvate carboxylase; a pyruvate carboxylase; a 6-phosphogluconate dehydrogenase; a transketolase; a puridine nucleotide transhydrogenase; a formate dehydrogenase; a glyceraldehyde-3P-dehydrogenase; a malic enzyme; a glucose dehydrogenase; a glucose-6-phosphate dehydrogenase; a fructose 1,6 diphosphatase; a L-alanine dehydrogenase; a L-glutamate dehydrogenase; a L-glutamine synthetase; a lysine transporter; a dicarboxylate transporter; and/or a multidrug transporter.

The reactions of the pathways described herein can be performed in one or more cell (e.g., host cell) strains (a) naturally expressing one or more relevant enzymes, (b) genetically engineered to express one or more relevant enzymes, or (c) naturally expressing one or more relevant enzymes and genetically engineered to express one or more relevant enzymes. Alternatively, relevant enzymes can be extracted from any of the above types of host cells and used in a purified or semi-purified form. Extracted enzymes optionally can be immobilized to the floors and/or walls of appropriate reaction vessels. Moreover, such extracts include lysates (e.g. cell lysates), and partially purified lysates, that can be used as sources of relevant enzymes. In the methods provided by the document, all the steps can be performed in cells (e.g., host cells), all the steps can be performed using extracted enzymes, or some of the steps can be performed in cells and others can be performed using extracted enzymes. In any of the methods, the reaction may be a single step conversion in which one compound is directly converted to a different compound of interest (e.g., D-proline to 5-aminopentanoate), or the conversion may include two or more steps to convert one compound to a different compound.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and the drawings, and from the claims. The word “comprising” in the claims may be replaced by “consisting essentially of” or with “consisting of,” according to standard practice in patent law.

### DESCRIPTION OF DRAWINGS

FIG. 1 is a schematic of an exemplary biochemical pathway leading to D-proline using glutamic acid as a central metabolite.

FIG. 2 is a schematic of an exemplary biochemical pathway leading to glutarate using D-proline as a central precursor.

FIG. 3 is a schematic of an exemplary biochemical pathway leading to 5-aminopentanoate using D-proline as a central precursor.

FIG. 4 is a schematic of exemplary biochemical pathways leading to cadaverine using 5-aminopentanoate (also known as 5-aminovalerate), 5-hydroxypentanoate, 5-oxopentanoate, or 1,5-pentanediol as a central precursor.

FIG. 5 is a schematic of an exemplary biochemical pathway leading to 5-hydroxypentanoate using D-proline as a central precursor.

FIG. 6 is a schematic of an exemplary biochemical pathway leading to 1,5 pentanediol using 5-hydroxypentanoate as a central precursor.

FIG. 7 contains the amino acid sequences of a Mycobacterium marinum carboxylate reductase (see Genbank Accession No. ACC40567.1, SEQ ID NO: 1), a Mycobacterium smegmatis carboxylate reductase (see Genbank Accession No. ABK71854.1, SEQ ID NO: 2), a Segniliparus rugosus carboxylate reductase (see Genbank Accession No. EFV11917.1, SEQ ID NO: 3), a Mycobacterium massiliense carboxylate reductase (see Genbank Accession No. EIV11143.1, SEQ ID NO: 5), a Segniliparus rotundus carboxylate reductase (see Genbank Accession No. ADG98140.1, SEQ ID NO: 6), a Chromobacterium violaceum ω-transaminase (see Genbank Accession No. AAQ59697.1, SEQ ID NO: 7), a Pseudomonas aeruginosa ω-transaminase (see Genbank Accession No. AAG08191.1, SEQ ID NO: 8), a Pseudomonas syringae ω-transaminase (see Genbank Accession No. AAY39893.1, SEQ ID NO: 9), a Rhodobacter sphaeroides ω-transaminase (see Genbank Accession No. ABA81135.1, SEQ ID NO: 10), an Escherichia coli ω-transaminase (see Genbank Accession No. AAA57874.1, SEQ ID NO: 11), a Vibrio fluvialis ω-transaminase (See Genbank Accession No. AEA39183.1, SEQ ID NO: 12), a Bacillus subtilis phosphopantetheinyl transferase (see Genbank Accession No. CAA44858.1, SEQ ID NO:13), and a Nocardia sp. NRRL 5646 phosphopantetheinyl transferase (see Genbank Accession No. ABI83656.1, SEQ ID NO:4).

FIG. 8 is a bar graph of the percent conversion after 4 hours (of reaction) of pyruvate to L-alanine (mol/mol) as a measure of the ω-transaminase (SEQ ID NO: 7) activity for converting 5-aminopentanoate to glutarate semialdehyde relative to the empty vector control.

FIG. 9 is a bar graph of the percent conversion after 4 hours (of reaction) of L-alanine to pyruvate (mol/mol) as a measure of the ω-transaminase (SEQ ID NO:9) activity for converting 5-oxopentanoate to 5-aminopentanoate relative to the empty vector control.

FIG. 10 is a bar graph summarizing the percent conversion after 4 hours (of reaction) of pyruvate to L-alanine (mol/mol) as a measure of the ω-transaminase (SEQ ID NOs: 7-12) activity of the enzyme only controls (no substrate).

FIG. 11 is a bar graph summarizing the change in absorbance at 340 nm after 20 minutes (of reaction), which is a measure of the consumption of NADPH and activity of carboxylate reductases (SEQ ID NOs: 1-3 and 5) relative to the enzyme only controls (no substrate).

FIG. 12 is a bar graph of the change in absorbance at 340 nm after 20 minutes (of reaction), which is a measure of the consumption of NADPH and the activity of carboxylate reductases (SEQ ID NOs: 1-3, 5, and 6) for converting 5-hydroxypentanoate to 5-hydroxypentanal relative to the empty vector control.

FIG. 13 is a bar graph of the percent conversion after 4 hours (of reaction) of pyruvate to L-alanine (mol/mol) as a measure of the ω-transaminase (SEQ ID NOs: 7-12) activity for converting 5-aminopentanol to 5-oxopentanol relative to the empty vector control.

FIG. 14 is a bar graph of the percent conversion after 4 hours (of reaction) of pyruvate to L-alanine (mol/mol) as a measure of the ω-transaminase (SEQ ID NOs: 7-9 and 11) activity for converting cadaverine to 5-aminopentanal relative to the empty vector control.

FIG. 15 is a bar graph of the change in absorbance at 340 nm after 20 minutes (of reaction), which is a measure of the consumption of NADPH and activity of carboxylate reductase (SEQ ID NO:6) for converting glutarate semialdehyde to pentanedial relative to the empty vector control.

### DETAILED DESCRIPTION

This document provides enzymes, non-natural pathways, cultivation strategies, feedstocks, host microorganisms and attenuations to the host's biochemical network, which generates a five carbon chain backbone such as D-proline from central metabolites in which one or two terminal functional groups may be formed leading to the synthesis of one or more of glutaric acid, 5-aminopentanoic acid, cadaverine, 5-hydroxypentanoic acid, or 1,5-pentanediol. Glutarate semialdehyde can be produced as an intermediate to other products. As used herein, the term “central precursor” is used to denote any metabolite in any metabolic pathway shown herein leading to the synthesis of a C5 building block. The term “central metabolite” is used herein to denote a metabolite that is produced in all microorganisms to support growth.

Host microorganisms described herein can include endogenous pathways that can be manipulated such that one or more C5 building blocks can be produced. In an endogenous pathway, the host microorganism naturally expresses all of the enzymes catalyzing the reactions within the pathway. A host microorganism containing an engineered pathway does not naturally express all of the enzymes catalyzing the reactions within the pathway but has been engineered such that all of the enzymes within the pathway are expressed in the host.

The term “exogenous” as used herein with reference to a nucleic acid (or a protein) and a host refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a host once in the host. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

For example, depending on the host and the compounds produced by the host, one or more of the following enzymes may be expressed in the host including a glutamate 5-kinase, a D-proline reductase, a pyrroline 5-carboxylate reductase, a proline racemase, a an aldehyde dehydrogenase, a succinate semialdehyde dehydrogenase, a glutamate semialdehyde dehydrogenase, an alcohol dehydrogenase, a 5-oxovalerate dehydrogenase, a 6-oxohexanoate dehydrogenase, a 7-oxoheptanoate dehydrogenase, a ω transaminase, a reversible ω transaminase, a 6-hydroxyhexanoate dehydrogenase, a 5-hydroxypentanoate dehydrogenase, 4-hydroxybutyrate dehydrogenase, or a carboxylate reductase. In recombinant hosts expressing a carboxylate reductase, a phosphopantetheinyl transferase also can be expressed as it enhances activity of the carboxylate reductase.

In some embodiments, a recombinant host can include at least one exogenous nucleic acid encoding one or more of a glutamate 5-kinase, a glutamate semialdehyde dehydrogenase, a pyrroline 5-carboxylate reductase, and a proline racemase, and produce D-proline. In some embodiments, a recombinant host includes at least one exogenous nucleic acid encoding a D-proline reductase. Either of such hosts further can include one or more of a reversible ω-transaminase (e.g., a 5-aminovalerate transaminase) and a dehydrogenase such as an aldehyde dehydrogenase, a succinate semialdehyde dehydrogenase, a 5-oxovalerate dehydrogenase, a 6-oxohexanoate dehydrogenase, or a 7-oxoheptanoate dehydrogenase and produce glutarate semialdehyde and/or glutarate. For example, either of such hosts further can include a reversible ω-transaminase (e.g., a 5-aminovalerate transaminase) and produce glutarate semialdehyde. For example, either of such hosts further can include a reversible ω transaminase (e.g., a 5-aminovalerate transaminase) and a dehydrogenase such as an aldehyde dehydrogenase, a succinate semialdehyde dehydrogenase, a 5-oxovalerate dehydrogenase, a 6-oxohexanoate dehydrogenase, or a 7-oxoheptanoate dehydrogenase and produce glutarate.

In some embodiments, a recombinant host includes at least one exogenous nucleic acid encoding a reversible ω transaminase (e.g., a 5-aminovalerate transaminase) and a dehydrogenase such as an aldehyde dehydrogenase, a succinate semialdehyde dehydrogenase, a 5-oxovalerate dehydrogenase, a 6-oxohexanoate dehydrogenase, or a 7-oxoheptanoate dehydrogenase and produces glutarate.

In some embodiments, a recombinant host includes at least one exogenous nucleic acid encoding a D-proline reductase, reversible ω transaminase (e.g., a 5-aminovalerate transaminase) and a dehydrogenase such as an aldehyde dehydrogenase, a succinate semialdehyde dehydrogenase, a 5-oxovalerate dehydrogenase, a 6-oxohexanoate dehydrogenase, or a 7-oxoheptanoate dehydrogenase and produces glutarate.

In some embodiments, a recombinant host that produces D-proline can include at least one exogenous nucleic acid encoding a D-proline reductase, and further produce 5-aminopentanoate.

In some embodiments, a recombinant host producing 5-aminopentanoate includes at least one exogenous nucleic acid encoding a reversible ω-transaminase (e.g., a 5-aminovalerate transaminase) and further produces glutarate semialdehyde.

In some embodiments, a recombinant host that produces D-proline includes at least one exogenous nucleic acid encoding a D-proline reductase and a reversible ω-transaminase (e.g., a 5-aminovalerate transaminase) and further produces glutarate semialdehyde.

In some embodiments, a recombinant host producing 5-aminopentanoate includes at least one exogenous nucleic acid encoding a reversible ω-transaminase (e.g., a 5-aminovalerate transaminase) and a dehydrogenase such as a 4-hydroxybutyrate dehydrogenase, a 5-hydroxypentanoate dehydrogenase, a 6-hydroxyhexanoate dehydrogenase or an alcohol dehydrogenase, and further produces 5-hydroxypentanoate.

In some embodiments, a recombinant host producing D-proline can include at least one exogenous nucleic acid encoding a D-proline reductase, a reversible ω transaminase (e.g., a 5-aminovalerate transaminase), and a dehydrogenase such as a 4-hydroxybutyrate dehydrogenase, a 5-hydroxypentanoate dehydrogenase, a 6-hydroxyhexanoate dehydrogenase or an alcohol dehydrogenase, and further produce 5-hydroxypentanoate.

A recombinant host producing 5-aminopentanoate, 5-hydroxypentanoate, or glutarate semialdehyde can include one or more of an exogenous carboxylate reductase, an exogenous ω-transaminase, or an exogenous alcohol dehydrogenase, and one or more (e.g., one, two, or three) optional exogenous enzymes such as a D-proline reductase, a 5-aminovalerate transaminase, and a dehydrogenase, and produce cadaverine. In some embodiments, a recombinant host can include each of an exogenous carboxylate reductase and an exogenous ω-transaminase and produce cadaverine. In some embodiments, a recombinant host can include each of an exogenous carboxylate reductase, an exogenous ω-transaminase, and an exogenous D-proline reductase and produce cadaverine. In some embodiments, a recombinant host can include each of an exogenous carboxylate reductase, an exogenous ω-transaminase, an exogenous D-proline reductase, and an exogenous 5-aminovalerate transaminase and produce cadaverine. In some embodiments, a recombinant host can include each of an exogenous carboxylate reductase, an exogenous ω-transaminase, and an exogenous 5-aminovalerate transaminase and produce cadaverine. In some embodiments, a recombinant host can include each of an exogenous carboxylate reductase, an exogenous ω-transaminase, an exogenous D-proline reductase, an exogenous 5-aminovalerate transaminase, and an exogenous dehydrogenase such as 6-hydroxyhexanoate dehydrogenase, a 5-hydroxypentanoate dehydrogenase, or a 4-hydroxybutyrate dehydrogenase and produce cadaverine. In some embodiments, a recombinant host can include each of an exogenous carboxylate reductase, an exogenous ω-transaminase, an exogenous 5-aminovalerate transaminase, and an exogenous dehydrogenase such as 6-hydroxyhexanoate dehydrogenase, a 5-hydroxypentanoate dehydrogenase, or a 4-hydroxybutyrate dehydrogenase and produce cadaverine. In some embodiments, a recombinant host can include an exogenous carboxylate reductase, at least one exogenous ω-transaminase (e.g., one exogenous ω-transaminase or two different exogenous ω-transaminases), and an exogenous alcohol dehydrogenase. In some embodiments, a recombinant host can include an exogenous carboxylate reductase, at least one exogenous ω-transaminase, and an exogenous alcohol dehydrogenase and produce cadaverine. In some embodiments, a recombinant host can include an exogenous carboxylate reductase, at least one exogenous ω-transaminase, an exogenous alcohol dehydrogenase, an exogenous 5-aminovalerate transaminase, and an exogenous dehydrogenase such as 6-hydroxyhexanoate dehydrogenase, a 5-hydroxypentanoate dehydrogenase, or a 4-hydroxybutyrate dehydrogenase and produce cadaverine. In some embodiments, a recombinant host can include an exogenous carboxylate reductase, at least one exogenous ω-transaminase, an exogenous alcohol dehydrogenase, an exogenous D-proline reductase, an exogenous 5-aminovalerate transaminase, and an exogenous dehydrogenase such as 6-hydroxyhexanoate dehydrogenase, a 5-hydroxypentanoate dehydrogenase, or a 4-hydroxybutyrate dehydrogenase and produce cadaverine.

A recombinant host producing 5-hydroxypentanoic acid can include one or more of a carboxylate reductase and an alcohol dehydrogenase, and produce 1,5-pentanediol. A recombinant host producing 1,5-pentanediol can include at least one exogenous ω-transaminase (e.g., one exogenous ω-transaminase or two different exogenous ω-transaminases) and optional second and/or third exogenous alcohol dehydrogenases and produce cadaverine.

In any of the recombinant hosts, one or more (e.g., one, two, three, or four) of the following exogenous enzymes can be included: a glutamate 5-kinase, a glutamate semialdehyde dehydrogenase, a pyrroline 5-carboxylate reductase, and a proline racemase.

Within an engineered pathway, the enzymes can be from a single source, i.e., from one species or genus, or can be from multiple sources, i.e., different species or genera. Nucleic acids encoding the enzymes described herein have been identified from various organisms and are readily available in publicly available databases such as GenBank or EMBL.

Any of the enzymes described herein that can be used for production of one or more C5 building blocks can have at least 70% sequence identity (homology) (e.g., at least 75%, 80%, 85%, 90%, 95%, 97%, 98%, 99%, or 100%) to the amino acid sequence of the corresponding wild-type enzyme. It will be appreciated that the sequence identity can be determined on the basis of the mature enzyme (e.g., with any signal sequence removed) or on the basis of the immature enzyme (e.g., with any signal sequence included).

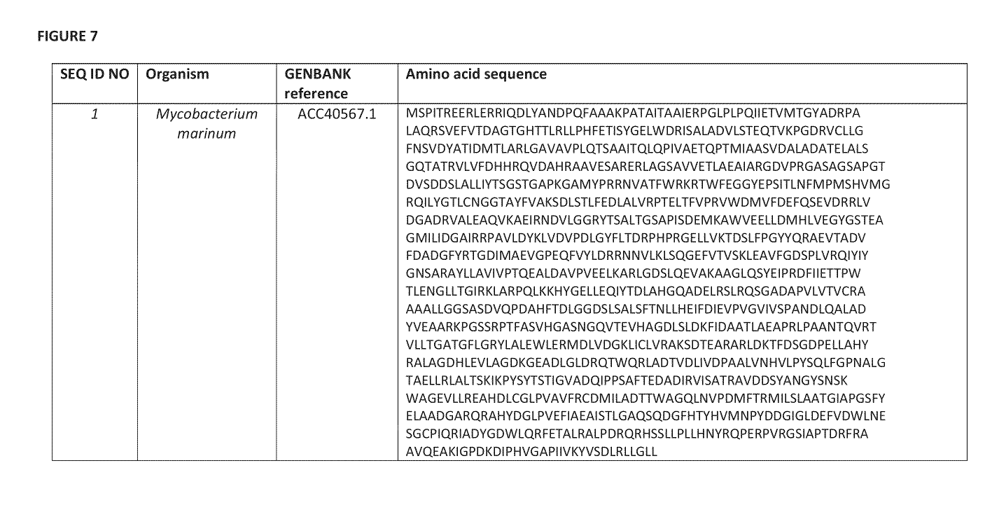

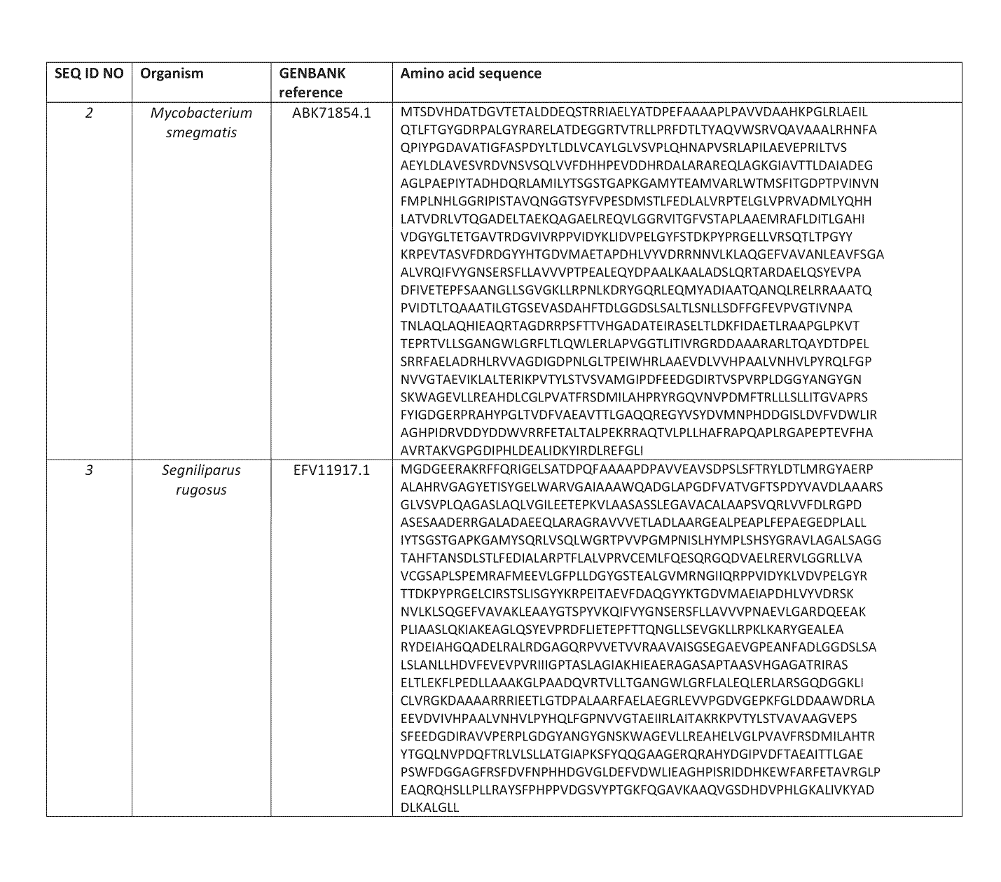

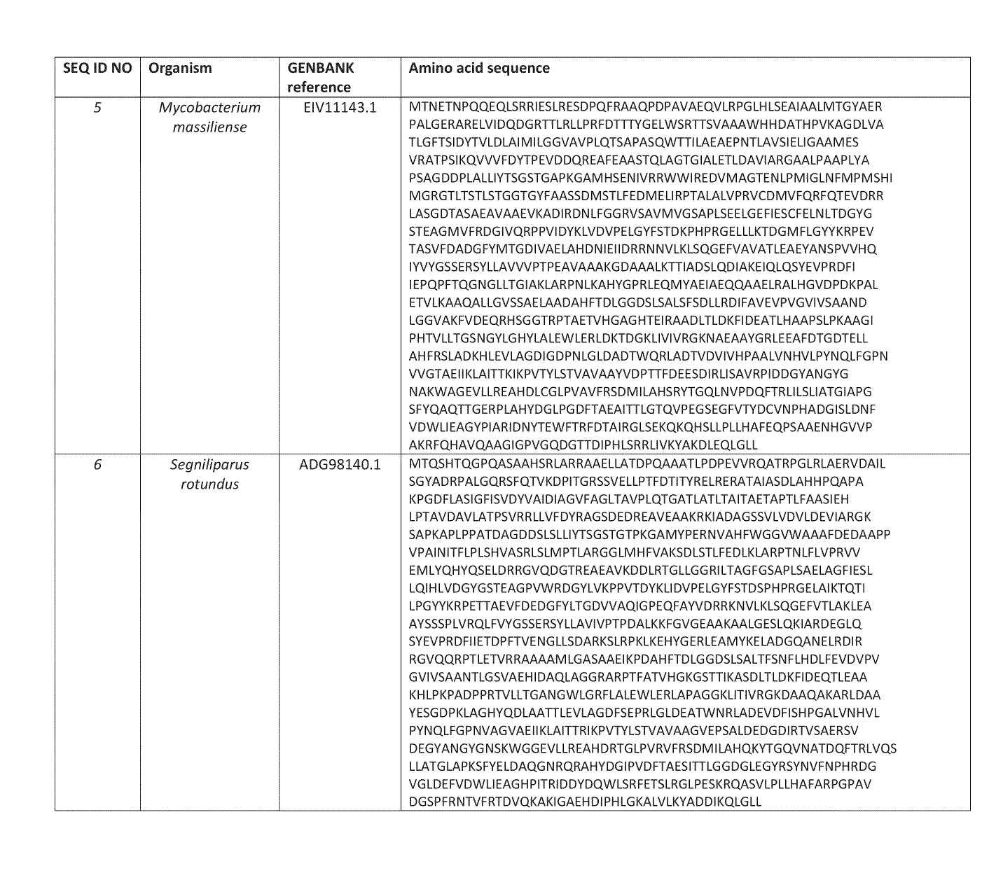

For example, a carboxylate reductase described herein can have at least 70% sequence identity (homology) (e.g., at least 75%, 80%, 85%, 90%, 95%, 97%, 98%, 99%, or 100%) to the amino acid sequence of a Mycobacterium marinum (see Genbank Accession No. ACC40567.1, SEQ ID NO: 1), a Mycobacterium smegmatis (see Genbank Accession No. ABK71854.1, SEQ ID NO: 2), a Segniliparus rugosus (see Genbank Accession No. EFV11917.1, SEQ ID NO: 3), a Mycobacterium massiliense (see Genbank Accession No. EIV 11143.1, SEQ ID NO: 5), or a Segniliparus rotundus (see Genbank Accession No. ADG98140.1, SEQ ID NO: 6) carboxylate reductase. See, FIG. 7.

For example, a ω-transaminase described herein can have at least 70% sequence identity (homology) (e.g., at least 75%, 80%, 85%, 90%, 95%, 97%, 98%, 99%, or 100%) to the amino acid sequence of a Chromobacterium violaceum (see Genbank Accession No. AAQ59697.1, SEQ ID NO: 7), a Pseudomonas aeruginosa (see Genbank Accession No. AAG08191.1, SEQ ID NO: 8), a Pseudomonas syringae (see Genbank Accession No. AAY39893.1, SEQ ID NO: 9), a Rhodobacter sphaeroides (see Genbank Accession No. ABA81135.1, SEQ ID NO: 10), an Escherichia coli (see Genbank Accession No. AAA57874.1, SEQ ID NO: 11), or a Vibrio fluvialis (see Genbank Accession No. AEA39183.1, SEQ ID NO: 12) ω-transaminase. Some of these ω-transaminases are diamine ω-transaminases. See, FIG. 7.

For example, a phosphopantetheinyl transferase described herein can have at least 70% sequence identity (homology) (e.g., at least 75%, 80%, 85%, 90%, 95%, 97%, 98%, 99%, or 100%) to the amino acid sequence of a Bacillus subtilis phosphopantetheinyl transferase (see Genbank Accession No. CAA44858.1, SEQ ID NO:13) or a Nocardia sp. NRRL 5646 phosphopantetheinyl transferase (see Genbank Accession No. ABI83656.1, SEQ ID NO:4). See FIG. 7.

The percent identity (homology) between two amino acid sequences can be determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology (identity), then the designated output file will present those regions of homology as aligned sequences. If the two compared sequences do not share homology (identity), then the designated output file will not present aligned sequences. Similar procedures can be following for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the enzymes described herein can also be used in the methods of the document. The term “functional fragment” as used herein refers to a peptide fragment of a protein that has at least 25% (e.g., at least: 30%; 40%; 50%; 60%; 70%; 75%; 80%; 85%; 90%; 95%; 98%; 99%; 100%; or even greater than 100%) of the activity of the corresponding mature, full-length, wild-type protein. The functional fragment can generally, but not always, be comprised of a continuous region of the protein, wherein the region has functional activity.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagglutinin (HA), glutathione-S-transferase (GST), or maltosebinding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

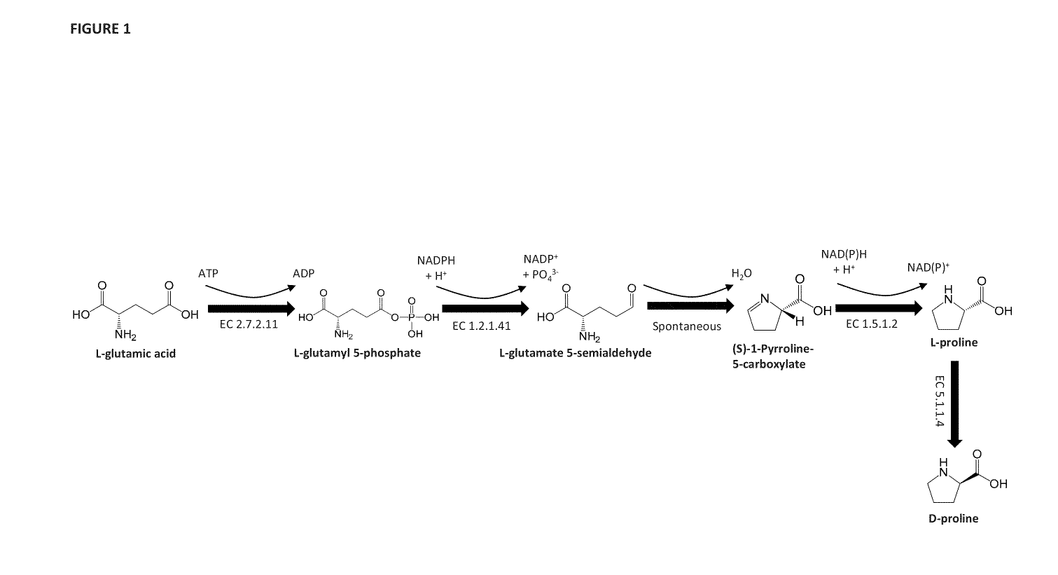

Engineered hosts can naturally express none or some (e.g., one or more, two or more, three or more, four or more, five or more, or six or more) of the enzymes of the pathways described herein in FIG. 1, 2, 3, 4, 5, or 6. Thus, a pathway within an engineered host can include all exogenous enzymes, or can include both endogenous and exogenous enzymes. Endogenous genes of the engineered hosts also can be disrupted to prevent the formation of undesirable metabolites or prevent the loss of intermediates in the pathway through other enzymes acting on such intermediates. Engineered hosts can be referred to as recombinant hosts or recombinant host cells. As described herein recombinant hosts can include nucleic acids encoding one or more of a reductase, racemase, kinase, dehydrogenase, or ω-transaminase as described herein.

In addition, the production of one or more C5 building blocks can be performed in vitro using the isolated enzymes described herein, using a lysate (e.g., a cell lysate) from a host microorganism as a source of the enzymes, or using a plurality of lysates from different host microorganisms as the source of the enzymes.

### Enzymes Generating the Terminal Carboxyl Groups in the Biosynthesis of a C5 Building Block

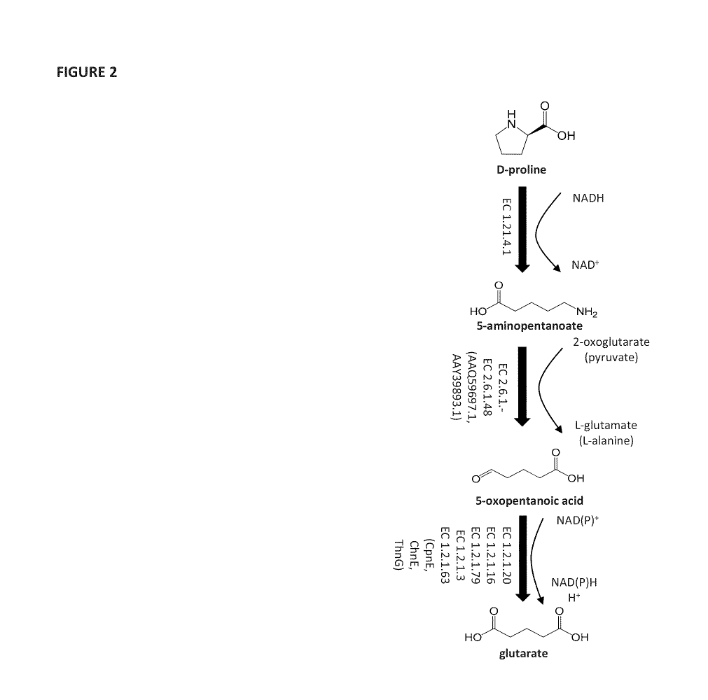

As depicted in FIG. 2, a terminal carboxyl group can be enzymatically formed using an aldehyde dehydrogenase, a glutarate semialdehyde dehydrogenase, a succinate semialdehyde dehydrogenase, a 7-oxoheptanoate dehydrogenase, a 6-oxohexanoate dehydrogenase, a 5-oxopentanoate dehydrogenase, or a reversible ω-transaminase.

In some embodiments, the first terminal carboxyl group leading to the synthesis of glutarate semialdehyde is formed by a reversible transaminase such as a 5-aminovalerate transaminase classified, for example, under EC 2.6.1.48, such as the reversible 5-aminovalerate transaminase obtained from Clostridium viride. See, for example, FIGS. 2 and 5 and SEQ ID NOs: 7 and 9. The reversible 5-aminovalerate transaminase from Clostridium viride has demonstrated analogous activity for the conversion of 6-aminohexanoate to adipate semialdehyde (Barker et al., J. Biol. Chem., 1987, 262(19), 8994-9003).

In some embodiments, the second terminal carboxyl group leading to the synthesis of glutaric acid is enzymatically formed by an aldehyde dehydrogenase classified, for example, under EC 1.2.1.3 (see, Guerrillot & Vandecasteele, Eur. J. Biochem., 1977, 81, 185-192). See, FIG. 2.

In some embodiments, the second terminal carboxyl group leading to the synthesis of glutaric acid is enzymatically formed by a dehydrogenase classified under EC 1.2.1.- such as a glutarate semialdehyde dehydrogenase classified, for example, under EC 1.2.1.20, a succinate-semialdehyde dehydrogenase classified, for example, under EC 1.2.1.16 or EC 1.2.1.79, or an aldehyde dehydrogenase classified under EC 1.2.1.3. For example a dehydrogenase classified under EC 1.2.1.- can be a 5-oxovalerate dehydrogenase such as the gene product of CpnE, a 6-oxohexanoate dehydrogenase (e.g., the gene product of ChnE from Acinetobacter sp.), or a 7-oxoheptanoate dehydrogenase (e.g., the gene product of ThnG from Sphingomonas macrogolitabida) (Iwaki et al., Appl. Environ. Microbiol., 1999, 65(11), 5158-5162; López-Sánchez et al., Appl. Environ. Microbiol., 2010, 76(1), 110-118). For example, a 6-oxohexanoate dehydrogenase can be classified under EC 1.2.1.63 such as the gene product of ChnE. For example, a 7-oxoheptanoate dehydrogenase can be classified under EC 1.2.1.-.

### Enzymes Generating the Terminal Amine Groups in the Biosynthesis of a C5 Building Block

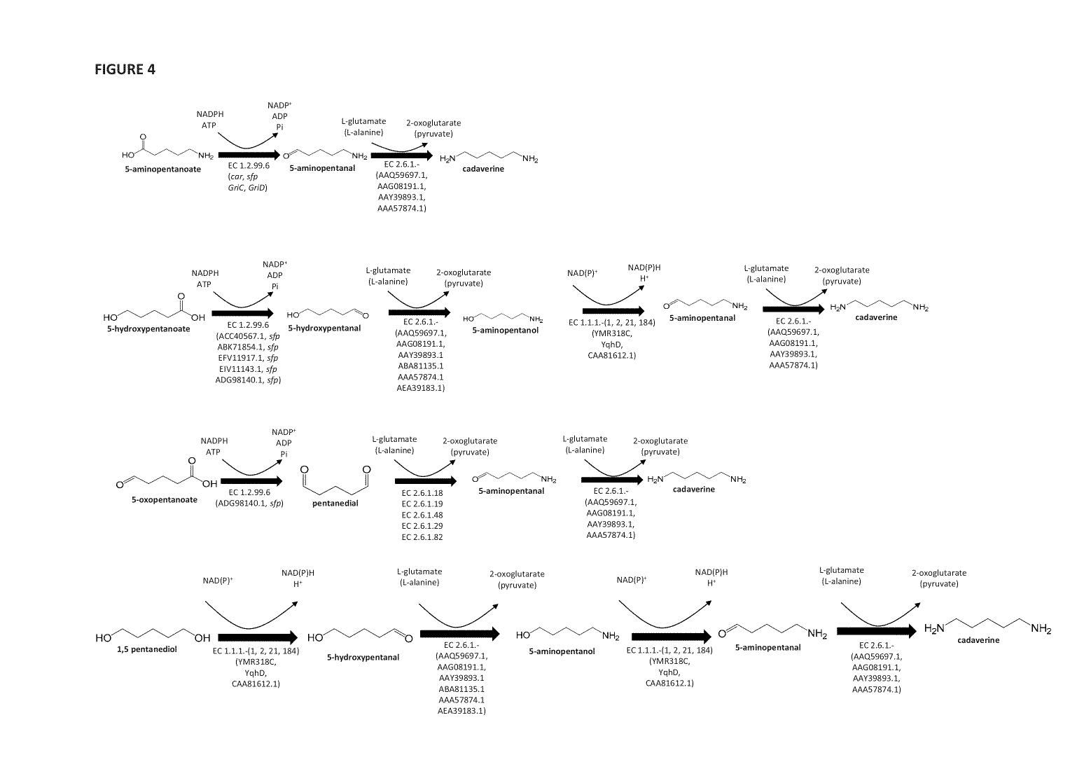

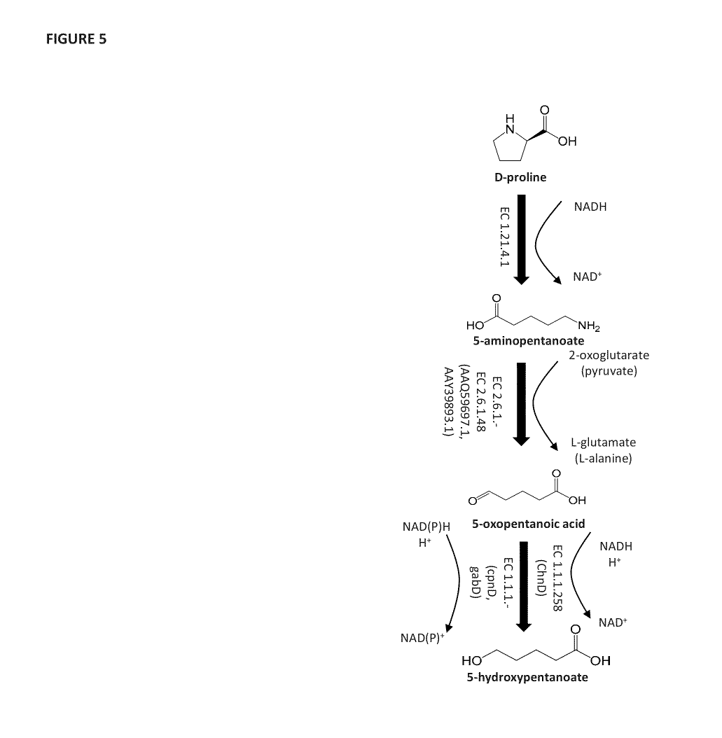

As depicted in FIG. 4 and FIG. 5, terminal amine groups can be enzymatically formed using a ω-transaminase or a D-proline reductase.

In some embodiments, one terminal amine group is enzymatically formed by a D-proline reductase classified, for example, under EC 1.21.4.1. See, FIGS. 2, 3, and 5.

In some embodiments, one terminal amine group leading to the synthesis of 5-aminopentanol or 5-aminopentanal can be enzymatically formed by a ω-transaminase classified, for example, under EC 2.6.1.18, EC 2.6.1.19, EC 2.6.1.29, EC 2.6.1.48, or EC 2.6.1.82 such as that obtained from Chromobacterium violaceum (Genbank Accession No. AAQ59697.1, SEQ ID NO: 7), Pseudomonas aeruginosa (Genbank Accession No. AAG08191.1, SEQ ID NO: 8), Pseudomonas syringae (Genbank Accession No. AAY39893.1, SEQ ID NO: 9), Rhodobacter sphaeroides (Genbank Accession No. ABA81135.1, SEQ ID NO: 10), Vibrio fluvialis (Genbank Accession No. AEA39183.1, SEQ ID NO: 12), Streptomyces griseus, or Clostridium viride. An additional ω-transaminase that can be used in the methods and hosts described herein is from Escherichia coli (Genbank Accession No. AAA57874.1, SEQ ID NO: 11). Some of the ω-transaminases classified, for example, under EC 2.6.1.29 or EC 2.6.1.82 are diamine ω-transaminases (e.g., SEQ ID NO:9). See, FIG. 4.

The reversible ω-transaminase from Chromobacterium violaceum (Genbank Accession No. AAQ59697.1, SEQ ID NO: 7) has demonstrated analogous activity accepting 6-aminohexanoic acid as amino donor, thus forming the first terminal amine group in adipate semialdehyde (Kaulmann et al., Enzyme and Microbial Technology, 2007, 41, 628-637).

The reversible 4-aminobubyrate:2-oxoglutarate transaminase from Streptomyces griseus has demonstrated analogous activity for the conversion of 6-aminohexanoate to adipate semialdehyde (Yonaha et al., Eur. J. Biochem., 1985, 146, 101-106).

In some embodiments, the second terminal amine group leading to the synthesis of cadaverine is enzymatically formed by a diamine transaminase. For example, the second terminal amino group can be enzymatically formed by a diamine transaminase classified, for example, under EC 2.6.1.29 or classified, for example, under EC 2.6.1.82, such as the gene product of YgjG from E. coli (Genbank Accession No. AAA57874.1, SEQ ID NO: 11).

The gene product of ygjG accepts a broad range of diamine carbon chain length substrates, such as putrescine, cadaverine and spermidine (Samsonova et al., BMC Microbiology, 2003, 3:2).

The diamine transaminase from E.coli strain B has demonstrated activity for 1,7 diaminoheptane (Kim, The Journal of Chemistry, 1964, 239(3), 783-786).

### Enzymes Generating the Terminal Hydroxyl Groups in the Biosynthesis of a C5 Building Block

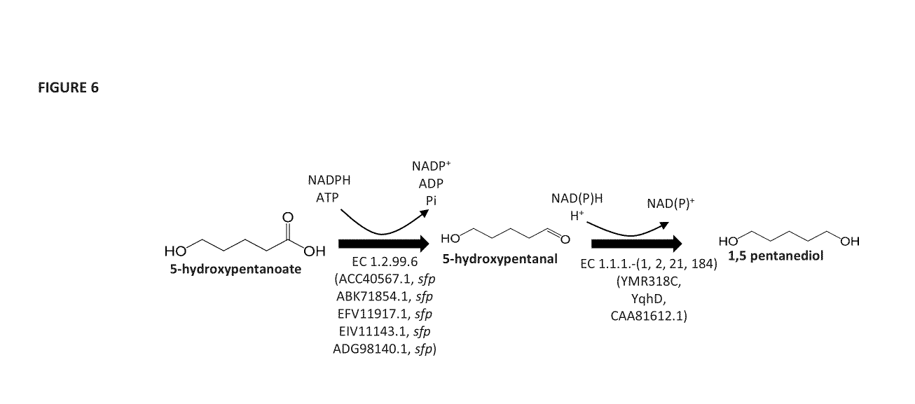

As depicted in FIGS. 5 and 6, a terminal hydroxyl group can be enzymatically formed using a dehydrogenase such as an alcohol dehydrogenase, a 6-hydroxyhexanoate dehydrogenase, a 5-hydroxypentanoate dehydrogenase, or a 4-hydroxybutyrate dehydrogenase.

For example, a terminal hydroxyl group leading to the synthesis of 5-hydroxypentanoate can be enzymatically formed by a dehydrogenase classified, for example, under EC 1.1.1.- such as a 6-hydroxyhexanoate dehydrogenase classified, for example, under EC 1.1.1.258 (e.g., the gene product of ChnD), a 5-hydroxypentanoate dehydrogenase classified, for example, under EC 1.1.1.- such as the gene product of CpnD (see, for example, Iwaki et al., 2002, Appl. Environ. Microbiol., 68(11):5671-5684), a 5-hydroxypentanoate dehydrogenase from Clostridium viride, or a 4-hydroxybutyrate dehydrogenase such as gabD (see, for example, Lütke-Eversloh & Steinbüchel, 1999, FEMS Microbiology Letters, 181(1):63-71). See, FIG. 5.

A terminal hydroxyl group leading to the synthesis of 1,5 pentanediol can be enzymatically formed by an alcohol dehydrogenase classified under EC 1.1.1.- (e.g., EC 1.1.1.1, 1.1.1.2, 1.1.1.21, or 1.1.1.184). See, FIG. 6.

### Biochemical Pathways

### Pathway to D-Proline

As depicted in FIG. 1, L-glutamic acid can be converted to L-glutamyl-phosphate by a glutamate 5-kinase classified, for example, under EC 2.7.2.11; followed by conversion of L-glutamyl-phosphate to L-glutamate semialdehyde by a dehydrogenase such as a glutamate-5-semialdehyde dehydrogenase classified, for example, under EC 1.2.1.41; followed by spontaneous conversion of L-glutamate semialdehyde to (S)-1-pyrroline-5-carboxylate; followed by conversion of (S)-1-pyrroline-5-carboxylate to L-proline by a pyrroline-5-carboxylate reductase classified, for example, under EC 1.5.1.2; followed by conversion of L-proline to D-proline by a proline racemase classified, for example, under EC 5.1.1.4.

### Pathway to Glutarate Using D-Proline as a Central Precursor

As depicted in FIG. 2, D-proline can be converted to 5-aminopentanoate (5-aminovaleric acid) by a D-proline reductase classified, for example, under EC 1.21.4.1; followed by conversion to 5-oxopentanoic acid (glutarate semialdehyde) by a ω-transaminase classified under EC 2.6.1- or a 5-aminovalerate transaminase classified, for example, under EC 2.6.1.48 (e.g., SEQ ID NO:7 or 9); followed by conversion to glutarate by a dehydrogenase classified under EC 1.2.1.- such as a glutarate semialdehyde dehydrogenase classified, for example, under EC 1.2.1.20, a succinate-semialdehyde dehydrogenase classified, for example, under EC 1.2.1.16 or EC 1.2.1.79, or an aldehyde dehydrogenase classified under EC 1.2.1.3. For example, a 5-oxovalerate dehydrogenase such as the gene product of CpnE, a 6-oxohexanoate dehydrogenase such as the gene product of ChnE, or a 7-oxoheptanoate dehydrogenase (e.g., the gene product of ThnG from Sphingomonas macrogolitabida) can be used to convert 5-oxopentanoic acid to glutarate.

### Pathway to 5-Aminopentanoate Using D-Proline as a Central Precursor

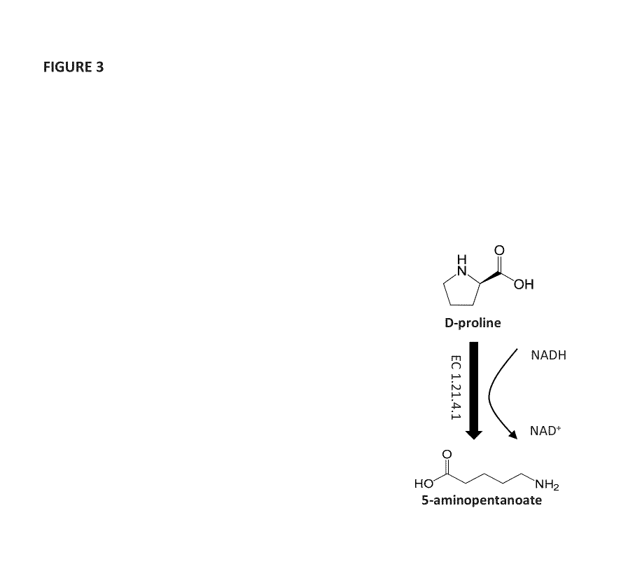

As depicted in FIGS. 2 and 3, D-proline can be converted to 5-aminopentanoate (5-aminovaleric acid) by a D-proline reductase classified, for example, under EC 1.21.4.1.

### Pathway Using 5-Aminopentanoate, 5-Hydroxypentanoate, Glutarate Semialdehyde or 1,5-Pentanediol as Central Precursor to Cadaverine

In some embodiments, cadaverine is synthesized from the central precursor 5-aminopentanoate (which can be produced, for example, in FIG. 3) by conversion of 5-aminopentanoate to 5-aminopentanal by a carboxylate reductase classified, for example, under EC 1.2.99.6 such as the gene product of car in combination with a phosphopantetheine transferase enhancer (e.g., encoded by a sfp gene from Bacillus subtilis or npt gene from Nocardia, SEQ ID NOs: 13 and 4, respectively) or the gene products of GriC and GriD from Streptomyces griseus (Suzuki et al., J. Antibiot., 2007, 60(6), 380-387); followed by conversion of 5-aminopentanal to cadaverine by a ω-transaminase (e.g., EC 2.6.1.18, EC 2.6.1.19, EC 2.6.1.48, EC 2.6.1.82 such as SEQ ID NOs:7-12). The carboxylate reductase can be obtained, for example, from Mycobacterium marinum (Genbank Accession No. ACC40567.1, SEQ ID NO: 1), Mycobacterium smegmatis (Genbank Accession No. ABK71854.1, SEQ ID NO: 2), Segniliparus rugosus (Genbank Accession No. EFV11917.1, SEQ ID NO: 3), Mycobacterium massiliense (Genbank Accession No. EIV11143.1, SEQ ID NO: 5), or Segniliparus rotundus (Genbank Accession No. ADG98140.1, SEQ ID NO: 6). See FIG. 4.

The carboxylate reductase encoded by the gene product of car and enhancer npt or sfp has broad substrate specificity, including terminal difunctional C4 and C5 carboxylic acids (Venkitasubramanian et al., Enzyme and Microbial Technology, 2008, 42, 130-137).

In some embodiments, cadaverine is synthesized from the central precursor 5-hydroxypentanoate (which can be produced as described in FIG. 5), by conversion of 5-hydroxypentanoate to 5-hydroxypentanal by a carboxylate reductase classified, for example, under EC 1.2.99.6 such as the gene product of car (see above) in combination with a phosphopantetheine transferase enhancer (see above); followed by conversion of 5-hydroxypentanal to 5-aminopentanol by a ω-transaminase classified, for example, under EC 2.6.1.18, EC 2.6.1.19, EC 2.6.1.29, EC 2.6.1.48, or EC 2.6.1.82 such as SEQ ID NOs:7-12, see above; followed by conversion to 5-aminopentanal by an alcohol dehydrogenase classified, for example, under EC 1.1.1.- (e.g., EC 1.1.1.1, EC 1.1.1.2, EC 1.1.1.21, or EC 1.1.1.184) such as the gene product of YMR318C or YqhD (Liu et al., Microbiology, 2009, 155, 2078-2085; Larroy et al., 2002, Biochem J., 361(Pt 1), 163-172; Jarboe, 2011, Appl. Microbiol. Biotechnol., 89(2), 249-257) or the protein having GenBank Accession No. CAA81612.1; followed by conversion to cadaverine by a ω-transaminase classified, for example, under EC 2.6.1.18, EC 2.6.1.19, EC 2.6.1.29, EC 2.6.1.48, or EC 2.6.1.82 such as SEQ ID NOs:7-12, see above. See FIG. 4.

In some embodiments, cadaverine is synthesized from the central precursor glutarate semialdehyde (also known as 5-oxopentanoate) by conversion of glutarate semialdehyde to pentanedial by a carboxylate reductase classified, for example, under EC 1.2.99.6 such as the gene product of car (see above) in combination with a phosphopantetheine transferase enhancer (see above); followed by conversion to 5-aminopentanal by a co-transaminase classified, for example, under EC 2.6.1.18, EC 2.6.1.19, or EC 2.6.1.48; followed by conversion to cadaverine by a ω-transaminase classified, for example, under EC 2.6.1.18, EC 2.6.1.19, EC 2.6.1.29, EC 2.6.1.48, or EC 2.6.1.82 such as SEQ ID NOs: 7-10. See FIG. 4.

In some embodiments, cadaverine is synthesized from the central precursor 1,5-pentanediol (which can be produced as described in FIG. 6), by conversion of 1,5-pentanediol to 5-hydroxypentanal by an alcohol dehydrogenase classified, for example, under EC 1.1.1.- such as EC 1.1.1.1, EC 1.1.1.2, EC 1.1.1.21, or EC 1.1.1.184) such as the gene product of YMR318C or YqhD (from E. coli, GenBank Accession No. AAA69178.1) (see, e.g., Liu et al., Microbiology, 2009, 155, 2078- 2085; Larroy et al., 2002, Biochem J., 361(Pt 1), 163-172; or Jarboe, 2011, Appl. Microbiol. Biotechnol., 89(2), 249-257) or the protein having GenBank Accession No. CAA81612.1 (from Geobacillus stearothermophilus); followed by conversion of 5-hydroxypentanal to 5-aminopentanol by a ω-transaminase classified, for example, under EC 2.6.1.18, EC 2.6.1.19, EC 2.6.1.29, EC 2.6.1.48, or EC 2.6.1.82 such as SEQ ID NOs:7-12, see above; followed by conversion to 5-aminopentanal by an alcohol dehydrogenase classified, for example, under EC 1.1.1.- (e.g., EC 1.1.1.1, EC 1.1.1.2, EC 1.1.1.21, or EC 1.1.1.184) such as the gene product of YMR318C or YqhD (Liu et al., Microbiology, 2009, 155, 2078-2085; Larroy et al., 2002, Biochem J., 361(Pt 1), 163-172; Jarboe, 2011, Appl. Microbiol. Biotechnol., 89(2), 249-257) or the protein having GenBank Accession No. CAA81612.1; followed by conversion to cadaverine by a ω-transaminase classified, for example, under EC 2.6.1.18, EC 2.6.1.19, EC 2.6.1.29, EC 2.6.1.48, or EC 2.6.1.82 such as SEQ ID NOs:7-10, see above. See FIG. 4.

### Pathway to 5-Hydroxypentanoate Using D-Proline as Central Precursor

As depicted in FIG. 5, D-proline can be converted to 5-aminopentanoate (5-aminovaleric acid) by a D-proline reductase classified, for example, under EC 1.21.4.1; followed by conversion to 5-oxopentanoic acid (glutarate semialdehyde) by a 5-aminovalerate transaminase classified, for example, under EC 2.6.1.48; followed by conversion to 5-hydroxypentanoate by a dehydrogenase classified, for example, under EC 1.1.1.- such as a 6-hydroxyhexanoate dehydrogenase classified, for example, under EC 1.1.1.258 (e.g., the gene product of ChnD), a 5-hydroxypentanoate dehydrogenase classified, for example, under EC 1.1.1.- such as the gene product of CpnD, or a 4-hydroxybutyrate dehydrogenase such as gabD.

### Pathway to 1,5-Pentanediol Using 5-Hydroxypentanoate as Central Precursor

As depicted in FIG. 6, 1,5 pentanediol can be synthesized from the central precursor 5-hydroxypentanoate by conversion of 5-hydroxypentanoate to 5-hydroxypentanal by a carboxylate reductase classified, for example, under EC 1.2.99.6 such as the gene product of car (see above) in combination with a phosphopantetheine transferase enhancer (see above); followed by conversion of 5-hydroxypentanal to 1,5 pentanediol by an alcohol dehydrogenase classified, for example, under EC 1.1.1.- such as EC 1.1.1.1, EC 1.1.1.2, EC 1.1.1.21, or EC 1.1.1.184) such as the gene product of YMR318C or YqhD (from E. coli, GenBank Accession No. AAA69178.1) (see, e.g., Liu et al., Microbiology, 2009, 155, 2078-2085; Larroy et al., 2002, Biochem J., 361(Pt 1), 163-172; or Jarboe, 2011, Appl. Microbiol. Biotechnol., 89(2), 249-257) or the protein having GenBank Accession No. CAA81612.1 (from Geobacillus stearothermophilus). See, FIG. 7 for the amino acid sequences of the above proteins.

### Cultivation Strategy

In some embodiments, a cultivation strategy entails either achieving an anaerobic, aerobic or micro-aerobic cultivation condition.

In some embodiments, a cyclical cultivation strategy entails alternating between achieving an anaerobic cultivation condition and achieving an aerobic cultivation condition.

In some embodiments, the cultivation strategy entails nutrient limitation such as nitrogen, phosphate or oxygen limitation.

In some embodiments, a cell retention strategy using, for example, ceramic hollow fiber membranes can be employed to achieve and maintain a high cell density during either fed-batch or continuous fermentation.

In some embodiments, the principal carbon source fed to the fermentation in the synthesis of one or more C5 building blocks can derive from biological or non-biological feedstocks.

In some embodiments, the biological feedstock can be or can derive from, monosaccharides, disaccharides, lignocellulose, hemicellulose, cellulose, lignin, levulinic acid and formic acid, triglycerides, glycerol, fatty acids, agricultural waste, condensed distillers' solubles, or municipal waste.

The efficient catabolism of crude glycerol stemming from the production of biodiesel has been demonstrated in several microorganisms such as Escherichia coli, Cupriavidus necator, Pseudomonas oleavorans, Pseudomonas putida and Yarrowia lipolytica (Lee et al., Appl. Biochem. Biotechnol., 2012, 166:1801-1813; Yang et al., Biotechnology for Biofuels, 2012, 5:13; Meijnen et al., Appl. Microbiol. Biotechnol., 2011, 90:885-893).

The efficient catabolism of lignocellulosic-derived levulinic acid has been demonstrated in several organisms such as Cupriavidus necator and Pseudomonas putida in the synthesis of 3-hydroxyvalerate via the precursor propanoyl-CoA (Jaremko and Yu, 2011, supra; Martin and Prather, J. Biotechnol., 2009, 139:61-67).

The efficient catabolism of lignin-derived aromatic compounds such as benzoate analogues has been demonstrated in several microorganisms such as Pseudomonas putida, Cupriavidus necator (Bugg et al., Current Opinion in Biotechnology, 2011, 22, 394-400; Pérez-Pantoja et al., FEMS Microbiol. Rev., 2008, 32, 736-794).

The efficient utilization of agricultural waste, such as olive mill waste water has been demonstrated in several microorganisms, including Yarrowia lipolytica (Papanikolaou et al., Bioresour. Technol., 2008, 99(7):2419-2428).

The efficient utilization of fermentable sugars such as monosaccharides and disaccharides derived from cellulosic, hemicellulosic, cane and beet molasses, cassava, corn and other agricultural sources has been demonstrated for several microorganism such as Escherichia coli, Corynebacterium glutamicum and Lactobacillus delbrueckii and Lactococcus lactis (see, e.g., Hermann et al, J. Biotechnol., 2003, 104:155-172; Wee et al., Food Technol. Biotechnol., 2006, 44(2):163-172; Ohashi et al., J. Bioscience and Bioengineering, 1999, 87(5):647-654).

The efficient utilization of furfural, derived from a variety of agricultural lignocellulosic sources, has been demonstrated for Cupriavidus necator (Li et al., Biodegradation, 2011, 22:1215-1225).

In some embodiments, the non-biological feedstock can be or can derive from natural gas, syngas, CO₂/H₂, methanol, ethanol, benzoate, non-volatile residue (NVR) or a caustic wash waste stream from cyclohexane oxidation processes, or terephthalic acid/isophthalic acid mixture waste streams.

The efficient catabolism of methanol has been demonstrated for the methylotrophic yeast Pichia pastoris.

The efficient catabolism of ethanol has been demonstrated for Clostridium kluyveri (Seedorf et al., Proc. Natl. Acad. Sci. USA, 2008, 105(6) 2128-2133).

The efficient catabolism of CO₂ and H₂, which may be derived from natural gas and other chemical and petrochemical sources, has been demonstrated for Cupriavidus necator (Prybylski et al., Energy, Sustainability and Society, 2012, 2:11).

The efficient catabolism of syngas has been demonstrated for numerous microorganisms, such as Clostridium ljungdahlii and Clostridium autoethanogenum (Köpke et al., Applied and Environmental Microbiology, 2011, 77(15):5467-5475).

The efficient catabolism of the non-volatile residue waste stream from cyclohexane processes has been demonstrated for numerous microorganisms, such as Delftia acidovorans and Cupriavidus necator (Ramsay et al., Applied and Environmental Microbiology, 1986, 52(1):152-156).

In some embodiments, the host microorganism is a prokaryote. For example, the prokaryote can be a bacterium from the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia such as Delftia acidovorans; from the genus Bacillus such as Bacillus subtillis; from the genus Lactobacillus such as Lactobacillus delbrueckii; or from the genus Lactococcus such as Lactococcus lactis. Such prokaryotes also can be a source of genes to construct recombinant host cells described herein that are capable of producing one or more C5 building blocks.

In some embodiments, the host microorganism is a eukaryote. For example, the eukaryote can be a filamentous fungus, e.g., one from the genus Aspergillus such as Aspergillus niger. Alternatively, the eukaryote can be a yeast, e.g., one from the genus Saccharomyces such as Saccharomyces cerevisiae; from the genus Pichia such as Pichia pastoris; or from the genus Yarrowia such as Yarrowia lipolytica; from the genus Issatchenkia such as Issathenkia orientalis; from the genus Debaryomyces such as Debaryomyces hansenii; from the genus Arxula such as Arxula adenoinivorans; or from the genus Kluyveromyces such as Kluyveromyces lactis. Such eukaryotes also can be a source of genes to construct recombinant host cells described herein that are capable of producing one or more C5 building blocks.

### Metabolic Engineering

The present document provides methods involving less than all the steps described for all the above pathways. Such methods can involve, for example, one, two, three, four, five, six, seven, eight, nine, ten, eleven, twelve or more of such steps. Where less than all the steps are included in such a method, the first, and in some embodiments the only, step can be any one of the steps listed.

Furthermore, recombinant hosts described herein can include any combination of the above enzymes such that one or more of the steps, e.g., one, two, three, four, five, six, seven, eight, nine, ten, or more of such steps, can be performed within a recombinant host. This document provides host cells of any of the genera and species listed and genetically engineered to express one or more (e.g., two, three, four, five, six, seven, eight, nine, 10, 11, 12 or more) recombinant forms of any of the enzymes recited in the document. Thus, for example, the host cells can contain exogenous nucleic acids encoding enzymes catalyzing one or more of the steps of any of the pathways described herein.

In addition, this document recognizes that where enzymes have been described as accepting CoA-activated substrates, analogous enzyme activities associated with [acp]-bound substrates exist that are not necessarily in the same enzyme class.

Also, this document recognizes that where enzymes have been described accepting (R)-enantiomers of substrate, analogous enzyme activities associated with (S)-enantiomer substrates exist that are not necessarily in the same enzyme class.

This document also recognizes that where an enzyme is shown to accept a particular co-factor, such as NADPH, or co-substrate, such as acetyl-CoA, many enzymes are promiscuous in terms of accepting a number of different co-factors or co-substrates in catalyzing a particular enzyme activity. Also, this document recognizes that where enzymes have high specificity for e.g., a particular co-factor such as NADH, an enzyme with similar or identical activity that has high specificity for the co-factor NADPH may be in a different enzyme class.

In some embodiments, the enzymes in the pathways outlined herein are the result of enzyme engineering via non-direct or rational enzyme design approaches with aims of improving activity, improving specificity, reducing feedback inhibition, reducing repression, improving enzyme solubility, changing stereo-specificity, or changing co-factor specificity.

In some embodiments, the enzymes in the pathways outlined here can be gene dosed, i.e., overexpressed, into the resulting genetically modified organism via episomal or chromosomal integration approaches.

In some embodiments, genome-scale system biology techniques such as Flux Balance Analysis can be utilized to devise genome scale attenuation or knockout strategies for directing carbon flux to a C5 building block.

Attenuation strategies include, but are not limited to; the use of transposons, homologous recombination (double cross-over approach), mutagenesis, enzyme inhibitors and RNAi interference.

In some embodiments, fluxomic, metabolomic and transcriptomal data can be utilized to inform or support genome-scale system biology techniques, thereby devising genome scale attenuation or knockout strategies in directing carbon flux to a C5 building block.

In some embodiments, the host microorganism's tolerance to high concentrations of a C5 building block can be improved through continuous cultivation in a selective environment.

In some embodiments, the host microorganism's endogenous biochemical network can be attenuated or augmented to (1) ensure the intracellular availability of glutamic acid or proline, (2) create a co-factor imbalance that may only be balanced via the formation of one or more C5 building blocks, (3) prevent degradation of central metabolites, central precursors leading to and including one or more C5 building blocks and/or (4) ensure efficient efflux from the cell.

In some embodiments requiring the intracellular availability of L-glutamate for C5 building block synthesis, the enzymes catalyzing anaplerotic reactions supplementing the citric acid cycle intermediates are amplified, such as a phosphoenolpyruvate carboxylase or a pyruvate carboxylase.

In some embodiments, where pathways require excess NADH co-factor for C5 building block synthesis, a recombinant formate dehydrogenase gene can be overexpressed in the host organism (Shen et al., 2011, supra).

In some embodiments, where pathways require excess NADH or NADPH co-factor for C5 building block synthesis, a recombinant transhydrogenase can be attenuated.

In some embodiments, carbon flux can be directed into the pentose phosphate cycle to increase the supply of NADPH by attenuating an endogenous glucose-6-phosphate isomerase (EC 5.3.1.9).

In some embodiments, carbon flux can be redirected into the pentose phosphate cycle to increase the supply of NADPH by overexpression a 6-phosphogluconate dehydrogenase and/or a transketolase (Lee et al., 2003, Biotechnology Progress, 19(5), 1444-1449).

In some embodiments, where pathways require excess NADPH co-factor in the synthesis of a C5 building block, a gene such as UdhA encoding a puridine nucleotide transhydrogenase can be overexpressed in the host organisms (Brigham et al., Advanced Biofuels and Bioproducts, 2012, Chapter 39, 1065-1090).

In some embodiments, where pathways require excess NADPH co-factor in the synthesis of a C5 Building Block, a recombinant glyceraldehyde-3-phosphate-dehydrogenase gene such as GapN can be overexpressed in the host organisms (Brigham et al., 2012, supra).

In some embodiments, where pathways require excess NADPH co-factor in the synthesis of a C5 building block, a recombinant malic enzyme gene such as maeA or maeB can be overexpressed in the host organisms (Brigham et al., 2012, supra).

In some embodiments, where pathways require excess NADPH co-factor in the synthesis of a C5 building block, a recombinant glucose-6-phosphate dehydrogenase gene such as zwf can be overexpressed in the host organisms (Lim et al., J. Bioscience and Bioengineering, 2002, 93(6), 543-549).

In some embodiments, where pathways require excess NADPH co-factor in the synthesis of a C5 building block, a recombinant fructose 1,6 diphosphatase gene such as fbp can be overexpressed in the host organisms (Becker et al., J. Biotechnol., 2007, 132:99-109).

In some embodiments, where pathways require excess NADPH co-factor in the synthesis of a C5 building block, endogenous triose phosphate isomerase (EC 5.3.1.1) can be attenuated.

In some embodiments, where pathways require excess NADPH co-factor in the synthesis of a C5 building block, a recombinant glucose dehydrogenase such as the gene product of gdh can be overexpressed in the host organism (Satoh et al., J. Bioscience and Bioengineering, 2003, 95(4):335-341).

In some embodiments, endogenous enzymes facilitating the conversion of NADPH to NADH can be attenuated, such as the NADH generation cycle that may be generated via inter-conversion of glutamate dehydrogenases classified under EC 1.4.1.2 (NADH-specific) and EC 1.4.1.4 (NADPH-specific).

In some embodiments, an endogenous glutamate dehydrogenase (EC 1.4.1.3) that utilizes both NADH and NADPH as co-factors can be attenuated.

In some embodiments using hosts that naturally accumulate polyhydroxyalkanoates, the endogenous polyhydroxyalkanoate synthase enzymes can be attenuated in the host strain.

In some embodiments, a L-alanine dehydrogenase can be overexpressed in the host to regenerate L-alanine from pyruvate as an amino donor for ω-transaminase reactions.

In some embodiments, a L-glutamate dehydrogenase, a L-glutamine synthetase, or a glutamate synthase can be overexpressed in the host to regenerate L-glutamate from 2-oxoglutarate as an amino donor for ω-transaminase reactions.

In some embodiments, enzymes such as pimeloyl-CoA dehydrogenase classified under, EC 1.3.1.62 and/or a glutaryl-CoA dehydrogenase classified, for example, under EC 1.3.8.6 that degrade central metabolites and central precursors leading to and including C5 building blocks can be attenuated.

In some embodiments, endogenous enzymes activating C5 building blocks via Coenzyme A esterification such as CoA-ligases (e.g., a glutaryl-CoA synthetase) classified under, for example, EC 6.2.1.6 can be attenuated.

In some embodiments, the efflux of a C5 building block across the cell membrane to the extracellular media can be enhanced or amplified by genetically engineering structural modifications to the cell membrane or increasing any associated transporter activity for a C5 building block.

The efflux of cadaverine can be enhanced or amplified by overexpressing broad substrate range multidrug transporters such as Blt from Bacillus subtilis (Woolridge et al., 1997, J. Biol. Chem., 272(14):8864-8866); AcrB and AcrD from Escherichia coli (Elkins & Nikaido, 2002, J. Bacteriol., 184(23), 6490-6499), NorA from Staphylococcus aereus (Ng et al., 1994, Antimicrob Agents Chemother, 38(6), 1345-1355), or Bmr from Bacillus subtilis (Neyfakh, 1992, Antimicrob Agents Chemother, 36(2), 484-485).

The efflux of 5-aminopentanoate and cadaverine can be enhanced or amplified by overexpressing the solute transporters such as the lysE transporter from Corynebacterium glutamicum (Bellmann et al., 2001, Microbiology, 147, 1765-1774).

The efflux of glutaric acid can be enhanced or amplified by overexpressing a dicarboxylate transporter such as the SucE transporter from Corynebacterium glutamicum (Huhn et al., Appl. Microbiol. & Biotech., 89(2), 327-335).

### Producing C5 Building Blocks Using a Recombinant Host

Typically, one or more C5 building blocks can be produced by providing a host microorganism and culturing the provided microorganism with a culture medium containing a suitable carbon source as described above. In general, the culture media and/or culture conditions can be such that the microorganisms grow to an adequate density and produce a C5 building block efficiently. For large-scale production processes, any method can be used such as those described elsewhere (Manual of Industrial Microbiology and Biotechnology, 2n^{d} Edition, Editors: A. L. Demain and J. E. Davies, ASM Press; and Principles of Fermentation Technology, P. F. Stanbury and A. Whitaker, Pergamon). Briefly, a large tank (e.g., a 100 gallon, 200 gallon, 500 gallon, or more tank) containing an appropriate culture medium is inoculated with a particular microorganism. After inoculation, the microorganism is incubated to allow biomass to be produced. Once a desired biomass is reached, the broth containing the microorganisms can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within this second tank can be the same as, or different from, that used in the first tank.

Once transferred, the microorganisms can be incubated to allow for the production of a C5 building block. Once produced, any method can be used to isolate C5 building blocks. For example, C5 building blocks can be recovered selectively from the fermentation broth via adsorption processes. In the case of glutaric acid and 5- aminopentanoic acid, the resulting eluate can be further concentrated via evaporation, crystallized via evaporative and/or cooling crystallization, and the crystals recovered via centrifugation. In the case of cadaverine and 1,5-pentanediol, distillation may be employed to achieve the desired product purity.

### EXAMPLES

### Example 1

### Enzyme Activity of ω-Transaminase Using 5-Aminopentanoate as Substrate and Forming 5-Oxopentanoate

A sequence encoding an N-terminal His-tag was added to the genes from Chromobacterium violaceum and Pseudomonas syringae encoding the ω-transaminases of SEQ ID NOs: 7 and 9, respectively (see FIG. 7) such that N-terminal HIS tagged ω-transaminases could be produced. Each of the resulting modified genes was cloned into a pET21a expression vector under control of the T7 promoter and each expression vector was transformed into a BL21[DE3] E. coli host. The resulting recombinant E. coli strains were cultivated at 37° C. in a 250 mL shake flask culture containing 50 mL LB media and antibiotic selection pressure, with shaking at 230 rpm. Each culture was induced overnight at 16° C. using 1 mM IPTG.

The pellet from each induced shake flask culture was harvested via centrifugation. Each pellet was resuspended and lysed via sonication. The cell debris was separated from the supernatant via centrifugation and the cell free extract was used immediately in enzyme activity assays.

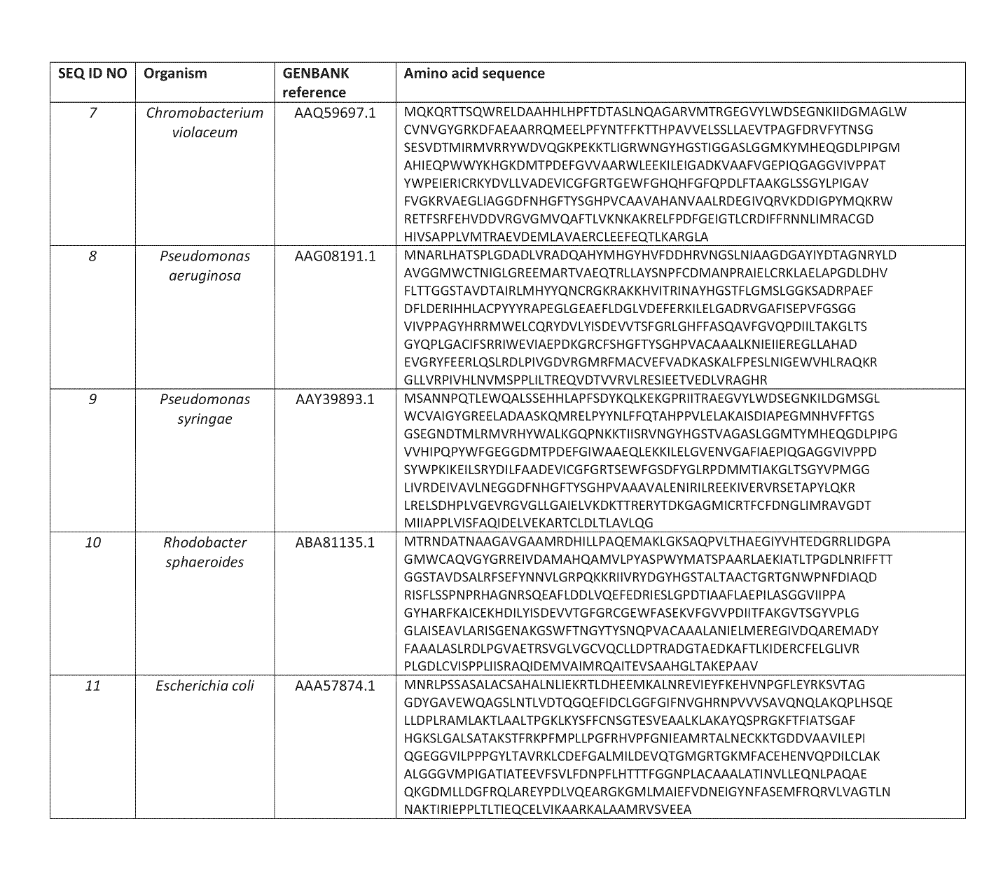

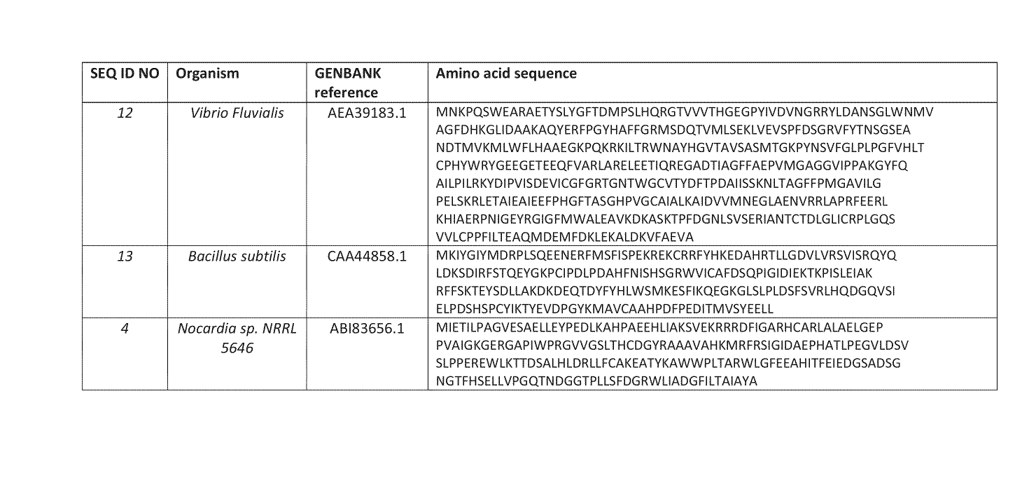

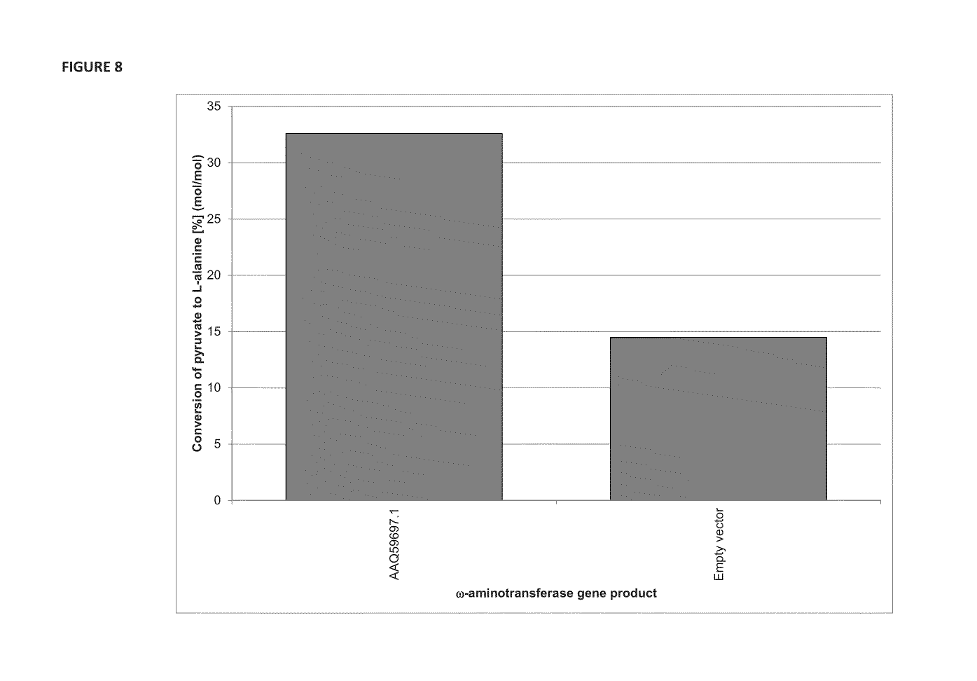

Enzyme activity assays in the forward direction (i.e., 5-aminopentanoate to glutarate semialdehyde) were performed in a buffer composed of a final concentration of 50 mM HEPES buffer (pH=7.5), 10 mM 5-aminopentanoate, 10 mM pyruvate and 100 μM pyridoxyl 5′ phosphate. Each enzyme activity assay reaction was initiated by adding cell free extract of the ω-transaminase gene product or the empty vector control to the assay buffer containing the 5-aminopentanoate and incubated at 25° C. for 4 h, with shaking at 250 rpm. The formation of L-alanine from pyruvate was quantified via RP-HPLC. The gene product of SEQ ID NO 7 accepted 5-aminopentanoate as substrate as confirmed against the empty vector control. See FIG. 8.

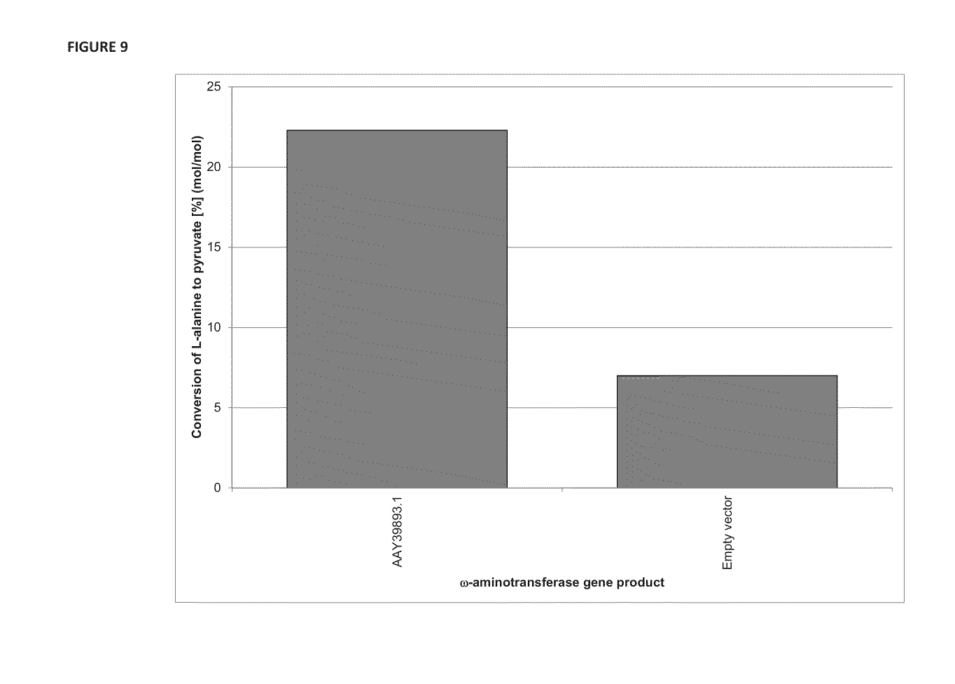

Enzyme activity in the reverse direction (i.e., glutarate semialdehyde to 5-aminopentanoate) were performed in a buffer composed of a final concentration of 50 mM HEPES buffer (pH=7.5), 10 mM glutarate semialdehyde, 10 mM L-alanine and 100 μM pyridoxyl 5′ phosphate. Each enzyme activity assay reaction was initiated by adding a cell free extract of the ω-transaminase gene product or the empty vector control to the assay buffer containing the glutarate semialdehyde and incubated at 25° C. for 4 h, with shaking at 250 rpm. The formation of pyruvate was quantified via RP-HPLC. The gene product of SEQ ID NO 9 accepted glutarate semialdehyde as substrate as confirmed against the empty vector control. See FIG. 9.

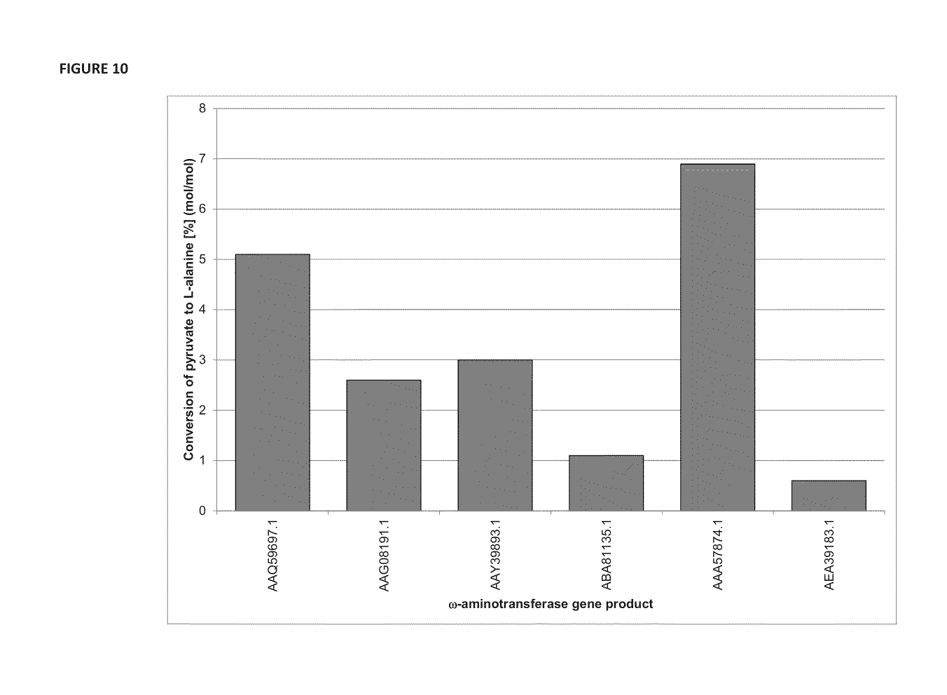

Each enzyme only control without 5-aminopentanoate demonstrated low base line conversion of pyruvate to L-alanine See FIG. 10.

Given the reversibility of the ω-transaminase activity, it can be concluded that the gene products of SEQ ID 7 and SEQ ID 9 accept 5-aminopentanoate as substrate and form 5-oxopentanoate.

### Example 2

### Enzyme Activity of Carboxylate Reductase Using 5-Hydroxypentanoate as Substrate and Forming 5-Hydroxypentanal

A sequence encoding a His-tag was added to the genes from Mycobacterium marinum, Mycobacterium smegmatis, Segniliparus rugosus, Mycobacterium massiliense, and Segniliparus rotundus that encode the carboxylate reductases of SEQ ID NOs: 1, 2, 3, 5 and 6, respectively (see FIG. 7) such that N-terminal HIS tagged carboxylate reductases could be produced. Each of the modified genes was cloned into a pET Duet expression vector alongside a sfp gene encoding a His-tagged phosphopantetheine transferase from Bacillus subtilis, both under control of the T7 promoter.

Each expression vector was transformed into a BL21[DE3] E. coli host and the resulting recombinant E. coli strains were cultivated at 37° C. in a 250 mL shake flask culture containing 50 mL LB media and antibiotic selection pressure, with shaking at 230 rpm. Each culture was induced overnight at 37° C. using an auto-induction media.

The pellet from each induced shake flask culture was harvested via centrifugation. Each pellet was resuspended and lysed via sonication. The cell debris was separated from the supernatant via centrifugation. The carboxylate reductases and phosphopantetheine transferase were purified from the supernatant using Ni-affinity chromatography, diluted 10-fold into 50 mM HEPES buffer (pH=7.5) and concentrated via ultrafiltration.

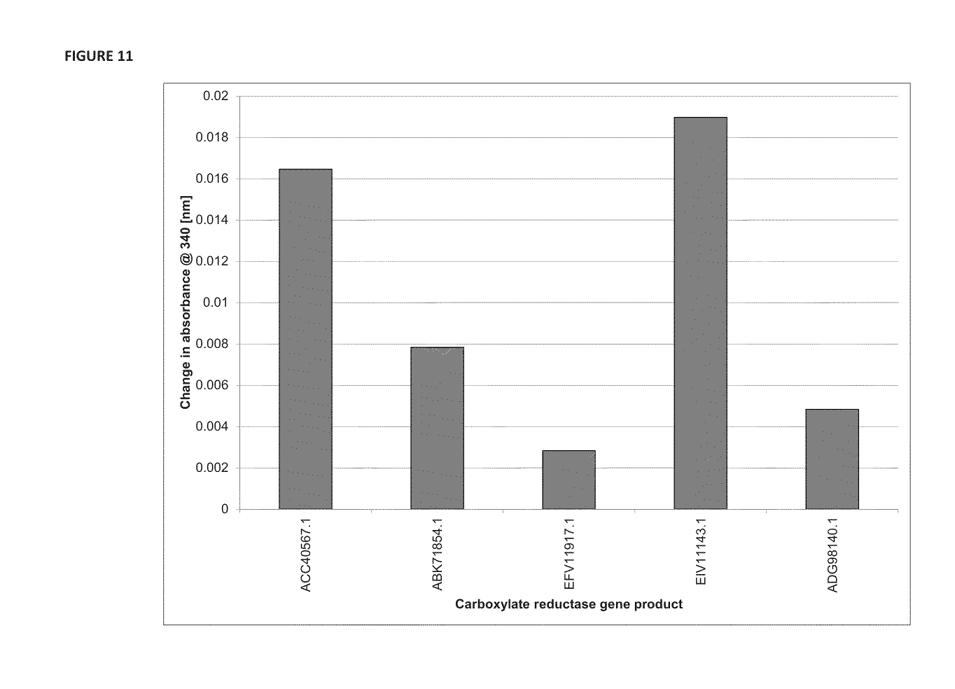

Enzyme activity (i.e., 5-hydroxypentanoate to 5-hydroxypentanal) assays were performed in triplicate in a buffer composed of a final concentration of 50 mM HEPES buffer (pH=7.5), 2 mM 5-hydroxypentanal, 10 mM MgCl₂, 1 mM ATP, and 1 mM NADPH. Each enzyme activity assay reaction was initiated by adding purified carboxylate reductase and phosphopantetheine transferase or the empty vector control to the assay buffer containing the 5-hydroxypentanoate and then incubated at room temperature for 20 min. The consumption of NADPH was monitored by absorbance at 340 nm. Each enzyme only control without 5-hydroxypentanoate demonstrated low base line consumption of NADPH. See FIG. 11.

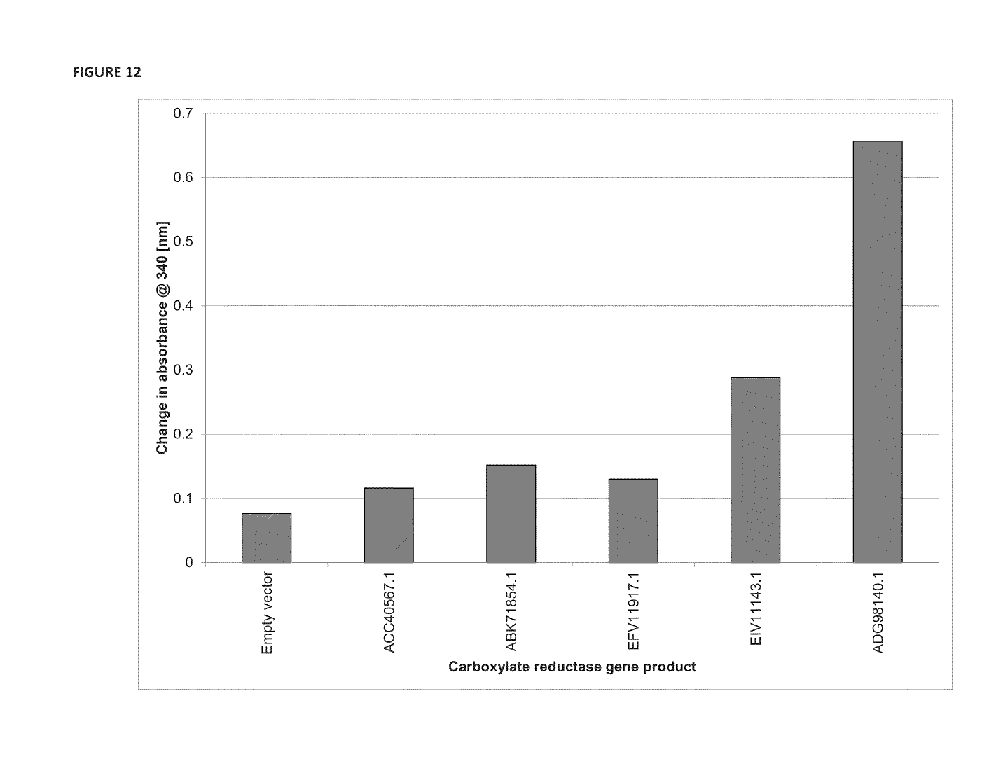

The gene products of SEQ ID NO 1, 2, 3, 5 and 6, enhanced by the gene product of sfp, accepted 5-hydroxypentanoate as substrate as confirmed against the empty vector control (see FIG. 12), and synthesized 5-hydroxypentanal.

### Example 3

### Enzyme Activity of ω-Transaminase for 5-Aminopentanol, Forming 5-Oxopentanol

A nucleotide sequence encoding an N-terminal His-tag was added to the Chromobacterium violaceum, Pseudomonas aeruginosa, Pseudomonas syringae, Rhodobacter sphaeroides, Escherichia coli and Vibrio Fluvialis genes encoding the ω-transaminases of SEQ ID NOs: 7-12 respectively (see FIG. 7) such that N-terminal HIS tagged ω-transaminases could be produced. The modified genes were cloned into a pET21a expression vector under the T7 promoter. Each expression vector was transformed into a BL21[DE3] E. coli host. Each resulting recombinant E. coli strain were cultivated at 37° C. in a 250 mL shake flask culture containing 50 mL LB media and antibiotic selection pressure, with shaking at 230 rpm. Each culture was induced overnight at 16° C. using 1 mM IPTG.

The pellet from each induced shake flask culture was harvested via centrifugation. Each pellet was resuspended and lysed via sonication. The cell debris was separated from the supernatant via centrifugation and the cell free extract was used immediately in enzyme activity assays.

Enzyme activity assays in the reverse direction (i.e., 5-aminopentanol to 5-oxopentanol) were performed in a buffer composed of a final concentration of 50 mM HEPES buffer (pH=7.5), 10 mM 5-aminopentanol, 10 mM pyruvate, and 100 μM pyridoxyl 5′ phosphate. Each enzyme activity assay reaction was initiated by adding cell free extract of the ω-transaminase gene product or the empty vector control to the assay buffer containing the 5-aminopentanol and then incubated at 25° C. for 4 h, with shaking at 250 rpm. The formation of L-alanine was quantified via RP-HPLC.

Each enzyme only control without 5-aminopentanol had low base line conversion of pyruvate to L-alanine See FIG. 10.

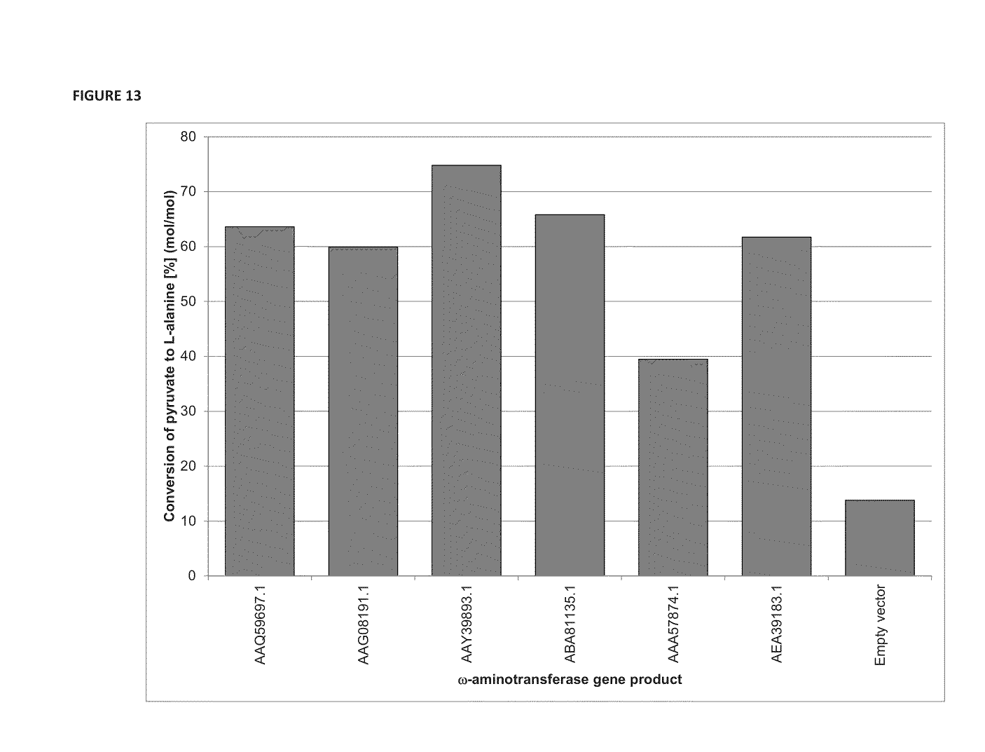

The gene products of SEQ ID NO 7-12 accepted 5-aminopentanol as substrate as confirmed against the empty vector control (see FIG. 13) and synthesized 5-oxopentanol as reaction product. Given the reversibility of the w-transaminase activity, it can be concluded that the gene products of SEQ ID 7-12 accept 5-oxopentanol as substrate and form 5-aminopentanol.

### Example 4

### Enzyme Activity of ω-Transaminase Using Cadaverine as Substrate and Forming 5-Aminopentanal

A sequence encoding an N-terminal His-tag was added to the Chromobacterium violaceum, Pseudomonas aeruginosa, Pseudomonas syringae and Escherichia coli genes encoding the ω-transaminases of SEQ ID NOs: 7, 8, 9 and 11, respectively (see FIG. 7) such that N-terminal HIS tagged ω-transaminases could be produced. The modified genes were cloned into a pET21a expression vector under the T7 promoter. Each expression vector was transformed into a BL21[DE3] E. coli host. Each resulting recombinant E. coli strain were cultivated at 37° C. in a 250 mL shake flask culture containing 50 mL LB media and antibiotic selection pressure, with shaking at 230 rpm. Each culture was induced overnight at 16° C. using 1 mM IPTG.

The pellet from each induced shake flask culture was harvested via centrifugation. Each pellet was resuspended and lysed via sonication. The cell debris was separated from the supernatant via centrifugation and the cell free extract was used immediately in enzyme activity assays.

Enzyme activity assays in the reverse direction (i.e., cadaverine to 5-aminopentanal) were performed in a buffer composed of a final concentration of 50 mM HEPES buffer (pH=7.5), 10 mM cadaverine, 10 mM pyruvate, and 100 μM pyridoxyl 5′ phosphate. Each enzyme activity assay reaction was initiated by adding cell free extract of the ω-transaminase gene product or the empty vector control to the assay buffer containing the cadaverine and then incubated at 25° C. for 4 h, with shaking at 250 rpm. The formation of L-alanine was quantified via RP-HPLC.

Each enzyme only control without cadaverine had low base line conversion of pyruvate to L-alanine See FIG. 10.

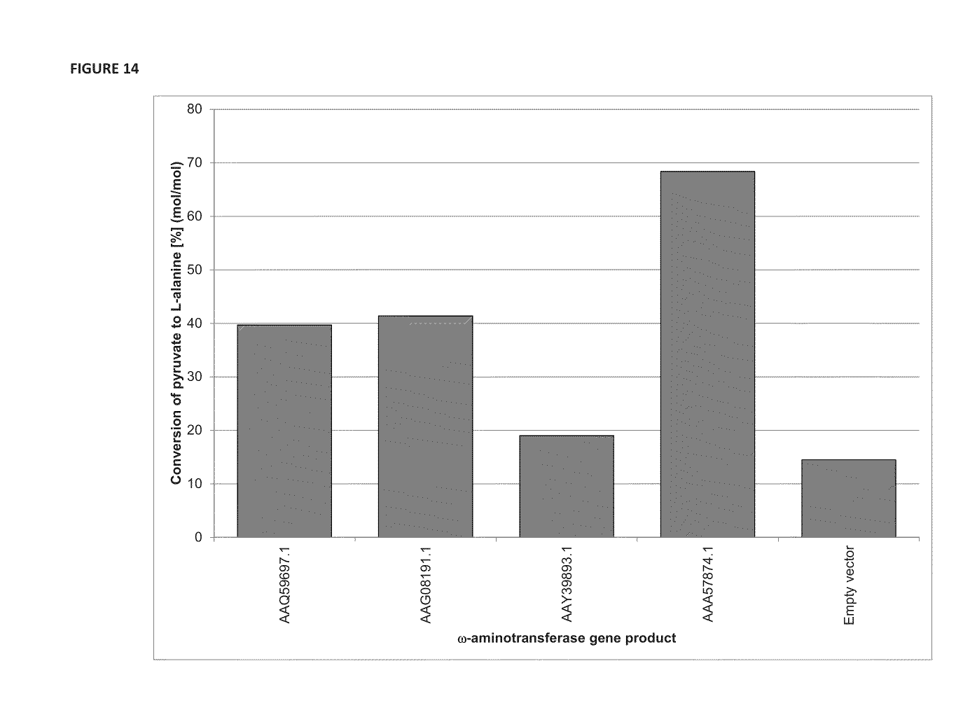

The gene products of SEQ ID NO 7, 8, 9 and 11 accepted cadaverine as substrate as confirmed against the empty vector control (see FIG. 14) and synthesized 5-aminopentanal as reaction product. Given the reversibility of the ω-transaminase activity, it can be concluded that the gene products of SEQ ID 7, 8, 9 and 11 accept 5-aminopentanal as substrate and form cadaverine.

### Example 5

### Enzyme Activity of Carboxylate Reductase Using Glutarate Semialdehyde as Substrate and Forming Pentanedial

The N-terminal His-tagged carboxylate reductase of SEQ ID NO 6 (see Example 2 and FIG. 7) was assayed using glutarate semialdehyde as substrate. The enzyme activity assay was performed in triplicate in a buffer composed of a final concentration of 50 mM HEPES buffer (pH=7.5), 2 mM glutarate semialdehyde, 10 mM MgCl₂, 1 mM ATP and 1 mM NADPH. The enzyme activity assay reaction was initiated by adding purified carboxylate reductase and phosphopantetheine transferase or the empty vector control to the assay buffer containing the glutarate semialdehyde and then incubated at room temperature for 20 min. The consumption of NADPH was monitored by absorbance at 340 nm. The enzyme only control without glutarate semialdehyde demonstrated low base line consumption of NADPH. See FIG. 11.

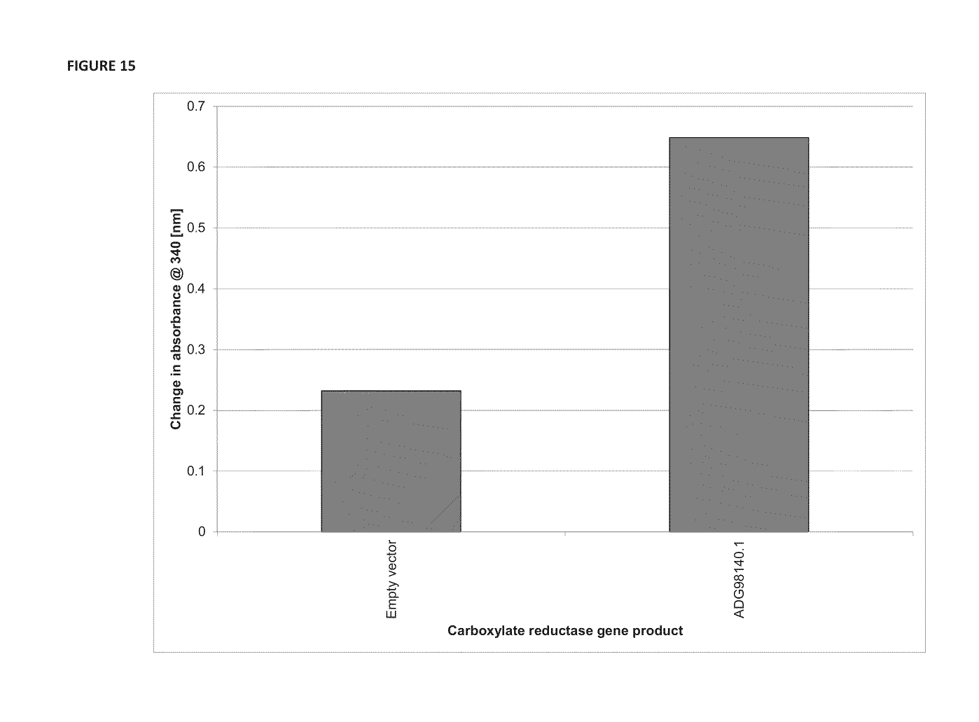

The gene product of SEQ ID NO 6, enhanced by the gene product of sfp, accepted glutarate semialdehyde as substrate as confirmed against the empty vector control (see FIG. 15) and synthesized pentanedial.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of producing a C5 building block selected from the group consisting of glutarate, 5-aminopentanoate, cadaverine, 5-hydroxpentanoate, and 1,5-pentanediol, said method comprising enzymatically synthesizing D-proline and enzymatically converting D-proline to said C5 building block in one or more enzymatic steps.

2. The method of claim 1, wherein D-proline is enzymatically synthesized from L-glutamic acid.

3. The method of claim 1, wherein D-proline is enzymatically converted to 5-aminopentanoate, glutarate, or 5-hydroxypentanoate.

4. The method of claim 3, wherein D-proline is enzymatically converted to 5-aminopentanoate using a D-proline reductase.

5. (canceled)

6. The method of claim 3, wherein D-proline is enzymatically converted to glutarate using a (i) D-proline reductase; (ii) a 5-aminovalerate transaminase; and (iii) a dehydrogenase selected from the group consisting of a glutarate semialdehyde dehydrogenase, a succinate-semialdehyde dehydrogenase, an aldehyde dehydrogenase, a 5-oxovalerate dehydrogenase, a 6-oxohexanoate dehydrogenase, and a 7-oxoheptanoate dehydrogenase.

7. (canceled)

8. The method of claim 3, wherein D-proline is enzymatically converted to 5-hydroxypentanoate using (i) a D-proline reductase; (ii) a 5-aminovalerate transaminase; and (iii) a dehydrogenase selected from the group consisting of a 6-hydroxyhexanoate dehydrogenase, a 5-hydroxypentanoate dehydrogenase, and a 4-hydroxybutyrate dehydrogenase.

9. The method of claim 6, wherein said 5-aminovalerate transaminase has at least 70% sequence identity to the amino acid sequence set forth in SEQ ID NO:7 or SEQ ID NO:9.

10. The method of claim 8, wherein 5-hydroxypentanoate is converted to cadaverine using a carboxylate reductase, an alcohol dehydrogenase, and a ω-transaminase.

11. The method of claim 10, wherein said ω-transaminase is classified under EC 2.6.1.18, EC 2.6.1.19, EC 2.6.1.29, 2.6.1.48, or EC 2.6.1.82.

12. The method of claim 8, wherein 5-hydroxypentanoate is converted to 1,5-pentanediol.

13. The method of claim 12, wherein 5-hydroxypentanoate is converted to 1,5-pentanediol using a carboxylate reductase and an alcohol dehydrogenase.

14. The method of claim 10, wherein said carboxylate reductase has at least 70% sequence identity to the amino acid sequence set forth in SEQ ID NO:1, SEQ ID NO:2, SEQ ID NO:3, SEQ ID NO:5, or SEQ ID NO:6.

15. The method of claim 12, wherein 1,5-pentanediol is enzymatically converted to cadaverine using an alcohol dehydrogenase and a ω-transaminase.

16. The method of claim 15, wherein said ω-transaminase has at least 70% sequence identity to an amino acid sequence set forth in SEQ ID NO: 7, SEQ ID NO: 8, SEQ ID NO: 9, SEQ ID NO: 10, SEQ ID NO: 11, or SEQ ID NO: 12.

17. The method of claim 3, wherein 5-aminopentanoate is enzymatically converted to cadaverine.

18. The method of claim 17, wherein 5-aminopentanoate is enzymatically converted to cadaverine using a carboxylate reductase and a ω-transaminase.

19. The method of claim 18, wherein said ω-transaminase has at least 70% sequence identity to an amino acid sequence set forth in SEQ ID NO: 7, SEQ ID NO: 8, SEQ ID NO: 9, or SEQ ID NO: 10.

20. The method of claim 1, wherein D-proline is enzymatically converted to cadaverine using a D-proline reductase, a 5-aminovalerate transaminase, an alcohol dehydrogenase, a carboxylate reductase, and a ω-transaminase.

21. The method of claim 20, wherein said 5-aminovalerate transaminase has at least 70% sequence identity to the amino acid sequence set forth in SEQ ID NO:7 or SEQ ID NO:9 and/or said carboxylate reductase has at least 70% sequence identity to the amino acid sequence set forth in SEQ ID NO:6.

22. (canceled)

23. The method of claim 1, wherein said method, in all or in part, is performed in a recombinant host by fermentation.

24. (canceled)

25. (canceled)

26. (canceled)

27. The method of claim 23, wherein the principal carbon source fed to the fermentation derives from a biological feedstock.

28. The method of claim 27, wherein the biological feedstock is, or derives from, monosaccharides, disaccharides, lignocellulose, hemicellulose, cellulose, lignin, levulinic acid, formic acid, triglycerides, glycerol, fatty acids, agricultural waste, condensed distillers' solubles, or municipal waste.

29. The method of claim 23, wherein the principal carbon source fed to the fermentation derives from a non-biological feedstock.

30. The method of claim 29, wherein the non-biological feedstock is, or derives from, natural gas, syngas, CO₂/H₂, methanol, ethanol, benzoate, non-volatile residue (NVR) caustic wash waste stream from cyclohexane oxidation processes, or terephthalic acid/isophthalic acid mixture waste streams.

31. The method of claim 23, wherein the host is a prokaryote.

32. The method of claim 31, wherein said prokaryote is from the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia acidovorans, from the genus Bacillus such as Bacillus subtillis; from the genes Lactobacillus such as Lactobacillus delbrueckii; from the genus Lactococcus such as Lactococcus lactis or from the genus Rhodococcus such as Rhodococcus equi.

33. The method of claim 23, wherein the host is a eukaryote.

34. The method of claim 33, wherein said eukaryote is from the genus Aspergillus such as Aspergillus niger; from the genus Saccharomyces such as Saccharomyces cerevisiae; from the genus Pichia such as Pichia pastoris; from the genus Yarrowia such as Yarrowia lipolytica, from the genus Issatchenkia such as Issathenkia orientalis, from the genus Debaryomyces such as Debaryomyces hansenii, from the genus Arxula such as Arxula adenoinivorans, or from the genus Kluyveromyces such as Kluyveromyces lactis.

35. The method of claim 23, wherein said host comprises one or more of the following attenuated enzymes: a polyhydroxyalkanoate synthase; a triose phosphate isomerase; a glucose-6-phosphate isomerase; a transhydrogenase; an NADH-specific glutamate dehydrogenase; a NADH/NADPH-utilizing glutamate dehydrogenase; a glutaryl-CoA dehydrogenase; or a glutaryl-CoA synthetase.

36. The method of claim 23, wherein said host overexpresses one or more genes encoding: a phosphoenolpyruvate carboxylase; a pyruvate carboxylase; a 6-phosphogluconate dehydrogenase; a transketolase; a puridine nucleotide transhydrogenase; aformate dehydrogenase; a glyceraldehyde-3P-dehydrogenase; a malic enzyme; a glucose dehydrogenase; a glucose-6-phosphate dehydrogenase; a fructose 1,6 diphosphatase; a L-alanine dehydrogenase; a L-glutamate dehydrogenase; a L-glutamine synthetase; a lysine transporter; a dicarboxylate transporter; and/or a multidrug transporter.

37. A recombinant host comprising at least one exogenous nucleic acid encoding (i) a D-proline reductase, (ii) a 5-aminovalerate transaminase, and (iii) a dehydrogenase, said host producing glutarate or 5-hydroxypentanoate.

38. The recombinant host of claim 37, wherein said host produces a) 5-hydroxypentanoate and said dehydrogenase is selected from the group consisting of a 6-hydroxyhexanoate dehydrogenase, a 5-hydroxypentanoate dehydrogenase, and a 4-hydroxybutyrate dehydrogenase or b) glutarate and said dehydrogenase is selected from the group consisting of glutarate semialdehyde dehydrogenase, a succinate-semialdehyde dehydrogenase, an aldehyde dehydrogenase, a 5-oxovalerate dehydrogenase, a 6-oxohexanoate dehydrogenase, and a 7-oxoheptanoate dehydrogenase.

39. (canceled)

40. The recombinant host of claim 38, said host further comprising an exogenous carboxylate reductase and an exogenous alcohol dehydrogenase, said host further producing 1,5-pentanediol.

41. The recombinant host of claim 38, said host further comprising an exogenous carboxylate reductase, an exogenous alcohol dehydrogenase, and at least one exogenous ω-transaminase, said host further producing cadaverine.

42. The recombinant host of claim 40, said host further comprising at least one exogenous ω-transaminase and an optional second and/or third exogenous alcohol dehydrogenase, said host further producing cadaverine.

43. The recombinant host of claim 41, wherein said at least one exogenous ω-transaminase has at least 70% sequence identity to an amino acid sequence set forth in SEQ ID NO: 7, SEQ ID NO: 8, SEQ ID NO: 9, SEQ ID NO: 10, SEQ ID NO:11, or SEQ ID NO:12.

44. The recombinant host of claim 41, wherein said host comprises two different exogenous ω-transaminases.

45. The recombinant host of claim 37, wherein said 5-aminovalerate transaminase has at least 70% sequence identity to the amino acid sequence set forth in SEQ ID NO:7 or SEQ ID NO:9.

46. A recombinant host comprising at least one exogenous nucleic acid encoding (i) a D-proline reductase, (ii) a carboxylate reductase, and (iii) a ω-transaminase, said host producing cadaverine.

47. The recombinant host of claim 46, wherein said ω-transaminase has at least 70% sequence identity to an amino acid sequence set forth in SEQ ID NO: 7, SEQ ID NO: 8, SEQ ID NO: 9, or SEQ ID NO: 10.

48. The host of claim 46, said host further comprising an exogenous 5-aminovalerate transaminase.

49. The host of claim 48, wherein said 5-aminovalerate transaminase has at least 70% sequence identity to the amino acid sequence set forth in SEQ ID NO:7 or SEQ ID NO:9.

50. The recombinant host of claim 46, wherein said carboxylate reductase has at least 70% sequence identity to the amino acid sequence set forth in SEQ ID NO:1, SEQ ID NO:2, SEQ ID NO:3, SEQ ID NO:5, or SEQ ID NO:6.

51. The recombinant host of claim 49, wherein said carboxylate reductase has at least 70% sequence identity to the amino acid sequence set forth in SEQ ID NO:6.

52. A recombinant host comprising a) at least one exogenous nucleic acid encoding (i) a 5-aminovalerate transaminase, and (ii) a dehydrogenase, said host producing glutarate or 5-hydroxypentanoate or b) at least one exogenous nucleic acid encoding (iii) a carboxylate reductase and (iv) a ω-transaminase, said host producing cadaverine.

53. (canceled)
